SEQUENCE LISTING (110 sequences)

SEQ ID NO: 1 - protein, 8 aa, Artificial Sequence
  synthetic
GFTFSSYG

SEQ ID NO: 2 - protein, 8 aa, Artificial Sequence
  synthetic
ISYDGSNK

SEQ ID NO: 3 - protein, 14 aa, Artificial Sequence
  synthetic
ARGGEYFYYYGLDV

SEQ ID NO: 4 - protein, 8 aa, Artificial Sequence
  synthetic
GYTFSNYG

SEQ ID NO: 5 - protein, 8 aa, Artificial Sequence
  synthetic
INTYNGNT

SEQ ID NO: 6 - protein, 19 aa, Artificial Sequence
  synthetic
ARDRGYSSSPYYYYYGMDV

SEQ ID NO: 7 - protein, 8 aa, Artificial Sequence
  synthetic
GYTFTNYG

SEQ ID NO: 8 - protein, 8 aa, Artificial Sequence
  synthetic
INAYNGNT

SEQ ID NO: 9 - protein, 8 aa, Artificial Sequence
  synthetic
GGSISTYY

SEQ ID NO: 10 - protein, 7 aa, Artificial Sequence
  synthetic
IYYSGST

SEQ ID NO: 11 - protein, 19 aa, Artificial Sequence
  synthetic
ARYDIVTGYPHYYYYVMDV

SEQ ID NO: 12 - protein, 7 aa, Artificial Sequence
  synthetic
QSVSSSS

SEQ ID NO: 13 - protein, 3 aa, Artificial Sequence
  synthetic
GAS

SEQ ID NO: 14 - protein, 9 aa, Artificial Sequence
  synthetic
QQDYNLPLT

SEQ ID NO: 15 - protein, 7 aa, Artificial Sequence
  synthetic
QSVSSSH

SEQ ID NO: 16 - protein, 9 aa, Artificial Sequence
  synthetic
QQDYNLTIT

SEQ ID NO: 17 - protein, 6 aa, Artificial Sequence
  synthetic
QGISNY

SEQ ID NO: 18 - protein, 3 aa, Artificial Sequence
  synthetic
AAS

SEQ ID NO: 19 - protein, 9 aa, Artificial Sequence
  synthetic
QQYNSYPFT

SEQ ID NO: 20 - protein, 6 aa, Artificial Sequence
  synthetic
QGISSA

SEQ ID NO: 21 - protein, 3 aa, Artificial Sequence
  synthetic
DAS

SEQ ID NO: 22 - protein, 9 aa, Artificial Sequence
  synthetic
QQFNNYPST

SEQ ID NO: 23 - protein, 6 aa, Artificial Sequence
  synthetic
QGIRND

SEQ ID NO: 24 - protein, 9 aa, Artificial Sequence
  synthetic
LQHNSYPYT

SEQ ID NO: 25 - protein, 6 aa, Artificial Sequence
  synthetic
QGIRNA

SEQ ID NO: 26 - protein, 9 aa, Artificial Sequence
  synthetic
LQDYNYPLT

SEQ ID NO: 27 - protein, 6 aa, Artificial Sequence
  synthetic
RGIRNA

SEQ ID NO: 28 - protein, 121 aa, Artificial Sequence
  synthetic
QVQLVESGGGVVQPGRSLRLSCAASGFTFSSYGMHWVRQAPGKGLEWVAVISYDGSNKNYVDSVKGRFTISRDNSKNTLYLQMNSLRAEDTAVYYCARGGEYFYYYGLDVWGQGTTVTVSS

SEQ ID NO: 29 - protein, 126 aa, Artificial Sequence
  synthetic
QVQLVQSGAEVKKPGASVKVSCKASGYTFSNYGISWVRQAPGQGLEWMGWINTYNGNTNYAQKLQGRVTMTTDTSTSTAYMALRGLRSDDTAVYYCARDRGYSSSPYYYYYGMDVWGQGTTVTVSS

SEQ ID NO: 30 - protein, 126 aa, Artificial Sequence; identical to SEQ ID NO: 29
  synthetic

SEQ ID NO: 31 - protein, 126 aa, Artificial Sequence; identical to SEQ ID NO: 29
  synthetic

SEQ ID NO: 32 - protein, 121 aa, Artificial Sequence; identical to SEQ ID NO: 28
  synthetic

SEQ ID NO: 33 - protein, 126 aa, Artificial Sequence; identical to SEQ ID NO: 29
  synthetic

SEQ ID NO: 34 - protein, 122 aa, Artificial Sequence
  synthetic
QVQLVESGGGVVQPGRSLRLSCAASGFTFSSYGMHWVRQAPGKGLEWVAVISYDGSNKNYVVDSVKGRFTISRDNSKNTLYLQMNSLRAEDTAVYYCARGGEYFYYYGLDVWGQGTTVTVSS

SEQ ID NO: 35 - protein, 126 aa, Artificial Sequence
  synthetic
QVQLVQSGAEVKKPGASVKVSCKASGYTFTNYGISWVRQAPGQGLEWMGWINAYNGNTNYAQKLQGRVTMTTVTYTSTAYMELRSLRSDDTAVYYCARDRGYSSSPYYYYYGMDVWGQGTTVTVSS

SEQ ID NO: 36 - protein, 125 aa, Artificial Sequence
  synthetic
QVQLQESGPGLVKPSETLSLTCTVSGGSISTYYWSWIRQPPGKGLEWIGYIYYSGSTNYNPSLKSRVTISVDTSKNQFSLKLSSVTAADTAVYYCARYDIVTGYPHYYYYVMDVWGQGTTVTVSS

SEQ ID NO: 37 - protein, 108 aa, Artificial Sequence
  synthetic
EIVMTQSPATLSLSPGERATLSCRASQSVSSSSLSWYQQKPGQAPRLLIYGASTRATGIPARFSGSGSGTDFTLTISSLQPEDFAVYYCQQDYNLPLTFGGGTKVEIK

SEQ ID NO: 38 - protein, 108 aa, Artificial Sequence
  synthetic
EIVMTQSPATLSLSPGERATLSCRASQSVSSSHLSWYQQKPGQAPRLLIYGASTRATGIPARFSGSGSGTDFTLTISSLQPEDFAVYYCQQDYNLTITFGQGTRLEIK

SEQ ID NO: 39 - protein, 108 aa, Artificial Sequence; identical to SEQ ID NO: 37
  synthetic

SEQ ID NO: 40 - protein, 107 aa, Artificial Sequence
  synthetic
DIQMTQSPSSLSASIGDRVTITCRASQGISNYLAWFQQKPGKAPKSLIYAASSLQSGVPSKFSGSGSGTDFTLTISSLQPEDFATYYCQQYNSYPFTFGPGTKVDIK

SEQ ID NO: 41 - protein, 107 aa, Artificial Sequence
  synthetic
AIQLTQSPSSLSASVGDRVTITCRAGQGISSALAWYQQKPGKAPKILIYDASSLESGVPSRFSGSGSGTDFTLTISSLQPEDFATYYCQQFNNYPSTFGQGTKLEIK

SEQ ID NO: 42 - protein, 107 aa, Artificial Sequence
  synthetic
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 43 - protein, 107 aa, Artificial Sequence; identical to SEQ ID NO: 40
  synthetic

SEQ ID NO: 44 - protein, 107 aa, Artificial Sequence
  synthetic
AIQMTQSPSSLSASVGDKVTITCRASQGIRNALGWYQQKPGTAPKLLIYAASSLQSGVPSRFSGSGSGTDFTLTISSLQPEDSATYYCLQDYNYPLTFGGGTKVEIK

SEQ ID NO: 45 - protein, 107 aa, Artificial Sequence
  synthetic
AIQMTQSPSSLSASVGDRVTITCRASRGIRNALGWYQQKPGTAPKLLIYAASSLQSGVPSRFSGSGSGTDFTLTISSLQPEDSATYYCLQDYNYPLTFGGGTKVEIK

SEQ ID NO: 46 - DNA, 363 bp, Artificial Sequence
  synthetic
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaaaactatgtagactccgtgaagggccgattcaccatctccagagacaattccaagaacacgctgtatctgcaaatgaacagcctgagagctgaggacacggctgtgtattactgtgcgagaggaggggagtacttctactattacggtttggacgtctggggccaagggaccacggtcaccgtctcctca

SEQ ID NO: 47 - DNA, 378 bp, Artificial Sequence
  synthetic
caggttcagctggtgcagtctggagctgaggtgaagaagcctggggcctcagtgaaggtctcctgcaaggcttctggttacaccttttccaattatggtatcagctgggtgcgacaggcccctggacaagggcttgagtggatgggatggatcaacacttacaatggtaacacaaactatgcacagaagctccagggcagagtcaccatgaccactgacacatccacgagcacagcctacatggcgctgaggggcctgagatctgacgacacggccgtgtattattgtgcgagagatagggggtatagcagcagcccttactactactactacggaatggacgtctggggccaagggaccacggtcaccgtctcctca

SEQ ID NO: 48 - DNA, 378 bp, Artificial Sequence; identical to SEQ ID NO: 47
  synthetic

SEQ ID NO: 49 - DNA, 378 bp, Artificial Sequence; identical to SEQ ID NO: 47
  synthetic

SEQ ID NO: 50 - DNA, 363 bp, Artificial Sequence; identical to SEQ ID NO: 46
  synthetic

SEQ ID NO: 51 - DNA, 378 bp, Artificial Sequence; identical to SEQ ID NO: 47
  synthetic

SEQ ID NO: 52 - DNA, 363 bp, Artificial Sequence; identical to SEQ ID NO: 46
  synthetic

SEQ ID NO: 53 - DNA, 378 bp, Artificial Sequence
  synthetic
caggttcagctggtgcagtctggagctgaggtgaagaagcctggagcctcagtgaaggtctcctgcaaggcttctggttacacctttaccaactatggtatcagctgggtgcgacaggcccctggacaagggcttgagtggatgggatggatcaacgcttacaatggtaacacaaactatgcacagaagctccagggcagagtcaccatgaccacagtcacatacacgagtacagcctacatggagctgaggagcctgagatctgacgacacggccgtgtattactgtgcgagagatagggggtatagcagcagcccttattactactactacggtatggacgtctggggccaagggaccacggtcaccgtctcctca

SEQ ID NO: 54 - DNA, 375 bp, Artificial Sequence
  synthetic
caggtgcagctgcaggagtcgggcccaggactggtgaagccttcggagaccctgtccctcacctgcactgtctctggtggctccatcagtacttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtttattactgtgcgaggtacgatattgtgactggttatcctcactactactactacgttatggacgtctggggccaagggaccacggtcaccgtctcctca

SEQ ID NO: 55 - DNA, 324 bp, Artificial Sequence
  synthetic
gaaattgtaatgacacagtctccagccaccctgtctttgtctccaggggaaagagccaccctctcctgcagggccagtcagagtgttagcagcagctccttatcctggtaccagcagaaacctgggcaggctcccaggctcctcatctatggtgcatccaccagggccactggcatcccagccaggttcagtggcagtgggtctgggacagacttcactctcaccatcagcagcctgcagcctgaagattttgcagtttattactgtcagcaggattataacttaccgctcactttcggcggagggaccaaggtggagatcaaa

SEQ ID NO: 56 - DNA, 324 bp, Artificial Sequence
  synthetic
gaaattgtaatgacacagtctccagccaccctgtctttgtctccaggggaaagagccaccctctcctgcagggccagtcagagtgttagcagcagccatttatcctggtaccagcagaaacctgggcaggctcccaggctcctcatctatggtgcatccaccagggccactggcatcccagccaggttcagtggcagtgggtctgggacagacttcactctcaccatcagcagcctgcagcctgaagattttgcagtttattattgtcagcaggattataatttaacgatcaccttcggccaagggacacgactggagattaaa

SEQ ID NO: 57 - DNA, 324 bp, Artificial Sequence; identical to SEQ ID NO: 55
  synthetic

SEQ ID NO: 58 - DNA, 321 bp, Artificial Sequence
  synthetic
gacatccagatgacccagtctccatcctcactgtctgcatctataggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttacccattcactttcggccctgggaccaaagtggatatcaaa

SEQ ID NO: 59 - DNA, 321 bp, Artificial Sequence
  synthetic
gccatccagttgacccagtctccatcctccctgtctgcatctgtaggagacagagtcaccatcacttgccgggcaggtcagggcattagcagtgctttagcctggtatcagcagaaaccagggaaagctcctaagatcctgatctatgatgcctccagtttggaaagtggggtcccatcaaggttcagcggcagtggatcggggacagatttcactctcaccatcagcagcctgcagcctgaagattttgcaacttattactgtcaacagtttaataattaccctagcacttttggccaggggaccaagctggagatcaaa

SEQ ID NO: 60 - DNA, 321 bp, Artificial Sequence
  synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaa

SEQ ID NO: 61 - DNA, 321 bp, Artificial Sequence; identical to SEQ ID NO: 58
  synthetic

SEQ ID NO: 62 - DNA, 321 bp, Artificial Sequence
  synthetic
gccatccagatgacccagtctccatcctccctgtctgcatctgtaggagacaaagtcaccatcacttgccgggcaagtcagggcattagaaatgctttaggctggtatcagcagaaaccaggaacagcccctaaactcctgatctatgctgcatccagtttacagagtggggtcccatcaaggttcagcggcagtggatctggcacagatttcactctcaccatcagcagcctgcagcctgaagattctgcaacttattactgtctacaagattacaattacccgctcactttcggcggagggaccaaggtggagatcaaa

SEQ ID NO: 63 - DNA, 321 bp, Artificial Sequence
  synthetic
gccatccagatgacccagtctccatcctccctgtctgcatctgtaggagacagagtcaccatcacttgccgggcaagtcggggcattagaaatgctttaggctggtatcagcagaaaccaggaacagcccctaaactcctgatctatgctgcatccagtttacagagtggggtcccatcaaggttcagcggcagtggatctggcacagatttcactctcaccatcagcagcctgcagcctgaagattctgcaacttattactgtctacaagattacaattacccgctcactttcggcggagggaccaaggtggagatcaaa

SEQ ID NO: 64 - protein, 327 aa, Artificial Sequence
  synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 326 aa, Artificial Sequence
  synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 66 - protein, 106 aa, Artificial Sequence
  synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 67 - protein, 106 aa, Artificial Sequence
  synthetic
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 68 - protein, 107 aa, Artificial Sequence [sequence omitted]

SEQ ID NO: 69 - protein, 162 aa, Artificial Sequence
  synthetic
THRTGLRSPDIPQDWVSFLRSFGQLTLCPRNGTVTGKWRGSHVVGLLTTLNFGDGPDRNKTRTFQATVLGSQMGLKGSSAGQLVLITARVTTERTAGTCLYFSAVPGILPSSQPPISCSEEGAGNATLSPRMGEECVSVWSHEGLVLTKLLTSEELALCGSR

SEQ ID NO: 70 - protein, 161 aa, Artificial Sequence
  synthetic
THTTGLRSPDIPQDWVSFLRSFGQLSLCPMNETVTGTWQGPHVVGLLTTLNFGDGPDRNKTQTFQAKIHGSQIGLTGSSAGESVLVTARVASGRTPGTCLYFSGVPKVLPSSQPPISCSEEGVGNATLSPVMGEECVRVWSHERLVLTELLTSEELALCGS

SEQ ID NO: 71 - protein, 25 aa, Artificial Sequence
  synthetic
QVQLQESGPGLVKPSETLSLTCTVS

SEQ ID NO: 72 - protein, 30 aa, Artificial Sequence
  synthetic
QVQLQESGPGLVKPSETLSLTCTVSGGSIS

SEQ ID NO: 73 - protein, 29 aa, Artificial Sequence
  synthetic
QVQLQESGPGLVKPSETLSLTCTVSGGSI

SEQ ID NO: 74 - protein, 7 aa, Artificial Sequence
  synthetic
GGSISTY

SEQ ID NO: 75 - protein, 10 aa, Artificial Sequence
  synthetic
GGSISTYYWS

SEQ ID NO: 76 - protein, 5 aa, Artificial Sequence
  synthetic
TYYWS

SEQ ID NO: 77 - protein, 6 aa, Artificial Sequence
  synthetic
STYYWS

SEQ ID NO: 78 - protein, 19 aa, Artificial Sequence
  synthetic
YWSWIRQPPGKGLEWIGYI

SEQ ID NO: 79 - protein, 14 aa, Artificial Sequence
  synthetic
WIRQPPGKGLEWIG

SEQ ID NO: 80 - protein, 11 aa, Artificial Sequence
  synthetic
WIRQPPGKGLE

SEQ ID NO: 81 - protein, 17 aa, Artificial Sequence
  synthetic
WSWIRQPPGKGLEWIGY

SEQ ID NO: 82 - protein, 5 aa, Artificial Sequence
  synthetic
YYSGS

SEQ ID NO: 83 - protein, 9 aa, Artificial Sequence
  synthetic
YIYYSGSTN

SEQ ID NO: 84 - protein, 16 aa, Artificial Sequence
  synthetic
YIYYSGSTNYNPSLKS

SEQ ID NO: 85 - protein, 12 aa, Artificial Sequence
  synthetic
WIGYIYYSGSTN

SEQ ID NO: 86 - protein, 41 aa, Artificial Sequence
  synthetic
TNYNPSLKSRVTISVDTSKNQFSLKLSSVTAADTAVYYCAR

SEQ ID NO: 87 - protein, 39 aa, Artificial Sequence
  synthetic
YNPSLKSRVTISVDTSKNQFSLKLSSVTAADTAVYYCAR

SEQ ID NO: 88 - protein, 32 aa, Artificial Sequence
  synthetic
RVTISVDTSKNQFSLKLSSVTAADTAVYYCAR

SEQ ID NO: 89 - protein, 37 aa, Artificial Sequence
  synthetic
YNPSLKSRVTISVDTSKNQFSLKLSSVTAADTAVYYC

SEQ ID NO: 90 - protein, 38 aa, Artificial Sequence
  synthetic
NYNPSLKSRVTISVDTSKNQFSLKLSSVTAADTAVYYC

SEQ ID NO: 91 - protein, 17 aa, Artificial Sequence
  synthetic
YDIVTGYPHYYYYVMDV

SEQ ID NO: 92 - protein, 18 aa, Artificial Sequence
  synthetic
ARYDIVTGYPHYYYYVMD

SEQ ID NO: 93 - protein, 11 aa, Artificial Sequence
  synthetic
WGQGTTVTVSS

SEQ ID NO: 94 - protein, 12 aa, Artificial Sequence
  synthetic
VWGQGTTVTVSS

SEQ ID NO: 95 - protein, 23 aa, Artificial Sequence
  synthetic
AIQMTQSPSSLSASVGDRVTITC

SEQ ID NO: 96 - protein, 29 aa, Artificial Sequence
  synthetic
AIQMTQSPSSLSASVGDRVTITCRASRGI

SEQ ID NO: 97 - protein, 26 aa, Artificial Sequence
  synthetic
AIQMTQSPSSLSASVGDRVTITCRAS

SEQ ID NO: 98 - protein, 11 aa, Artificial Sequence
  synthetic
RASRGIRNALG

SEQ ID NO: 99 - protein, 7 aa, Artificial Sequence
  synthetic
RNALGWY

SEQ ID NO: 100 - protein, 15 aa, Artificial Sequence
  synthetic
WYQQKPGTAPKLLIY

SEQ ID NO: 101 - protein, 9 aa, Artificial Sequence
  synthetic
QQKPGTAPK

SEQ ID NO: 102 - protein, 17 aa, Artificial Sequence
  synthetic
LGWYQQKPGTAPKLLIY

SEQ ID NO: 103 - protein, 7 aa, Artificial Sequence
  synthetic
AASSLQS

SEQ ID NO: 104 - protein, 10 aa, Artificial Sequence
  synthetic
LLIYAASSLQ

SEQ ID NO: 105 - protein, 32 aa, Artificial Sequence
  synthetic
GVPSRFSGSGSGTDFTLTISSLQPEDSATYYC

SEQ ID NO: 106 - protein, 33 aa, Artificial Sequence
  synthetic
SGVPSRFSGSGSGTDFTLTISSLQPEDSATYYC

SEQ ID NO: 107 - protein, 37 aa, Artificial Sequence
  synthetic
SSLQSGVPSRFSGSGSGTDFTLTISSLQPEDSATYYC

SEQ ID NO: 108 - protein, 8 aa, Artificial Sequence
  synthetic
LQDYNYPL

SEQ ID NO: 109 - protein, 10 aa, Artificial Sequence
  synthetic
FGGGTKVEIK

SEQ ID NO: 110 - protein, 11 aa, Artificial Sequence
  synthetic
TFGGGTKVEIK

(19) United States
(12) Patent Application Publication
(10) Pub. No.: US 2023/0039165 A1
(43) Pub. Date: Feb. 9, 2023

(54) IGFBP3 ANTIBODIES AND THERAPEUTIC USES THEREOF

(71) Applicant: ENTHERA S.R.L., Milan (IT)

(72) Inventors: Giovanni AMABILE, Milan (IT); Paolo FIORINA, Milan (IT); Francesca D'ADDIO, Milan (IT)

(21) Appl. No.: 17/778,814

(22) PCT Filed: Nov. 20, 2020
(86) PCT No.: PCT/EP2020/082890

§ 371 Date: May 20, 2022

(30) Foreign Application Priority Data
Nov. 21, 2019 (EP) 19210646.6
Apr. 1, 2020 (EP) 20167464.5

(51) Int. Cl. C07K 16/22; C12N 15/66; A61P 7/00

(52) U.S. Cl. CPC C07K 16/22; C12N 15/66; A61P 7/00; C07K 2317/92; C07K 2317/565; C07K 2317/76; C07K 2317/24

(57) ABSTRACT

The present invention relates to antibodies or antigen binding fragment thereof that binds specifically to IGFBP3. The antibody inhibits or reduces the binding of IGFBP3 to the TMEM219 receptor. The invention also relates to methods for their production, pharmaceutical compositions containing said antibodies, and uses thereof.

### TECHNICAL FIELD

The present invention relates to antibodies or antigen binding fragments thereof that bind specifically to human IGFBP3, methods for their production, pharmaceutical compositions containing said antibodies, and uses thereof.

### BACKGROUND ART

IGFBP3/TMEM219 Axis

The insulin-like growth factor binding proteins is a family of seven binding proteins which modulate the bioavailability of insulin-like growth factors (IGFs). Among them IGFBP3 is the most abundant, being present in almost all tissues, and has the higher affinity for IGFs; indeed, approximately 80-90% of IGFs are bound to IGFBP3 in a ternary complex with the acid labile subunit (ALS) (1).

In addition to its ability to regulate IGFs availability, IGFBP3 has also been shown to have IGF-independent functions (2). Indeed, it is able to associate with cell-surface proteins, cell-surface receptors with integral signaling capacity, intracellular and nuclear proteins (transcription factors) thus influencing cell growth and directly inducing apoptosis (2). Among death receptors, TMEM219, a single-span membrane protein, was shown high binding to IGFBP-3 (3). Binding of IGFBP3 to TMEM219 induces caspase-8-mediated apoptosis in a variety of cells, including cancer cells (i.e. prostate and breast) (3), but also stem cells (i.e. colonic stem cells)(4). Blocking or enhancing IGFBP3/TMEM219 axis with different strategies has been shown to respectively prevent or increase cell death. To the best of our knowledge there are no monoclonal antibodies against TMEM219 or IGFBP3 commercially available capable of preventing the IGFBP3/TMEM219 binding and halting the IGF-I independent and Caspase8-mediated, detrimental effects on target tissues/cells of binding of IGFBP3 to TMEM219.

IGFBP3/TMEM219 Axis in Diabetes

Type 1 (T1 D) and type 2 diabetes (T2D) are both characterized by a loss of beta cells, which results in a reduced secretion of insulin, failure to control blood glucose levels and hyperglycemia (5,6). Despite different etiological mechanisms, either autoimmune response in T1 D or insulin resistance/inflammation in T2D, both lead to a progressive reduction of beta cell mass. Indeed, it is becoming evident that the occurring autoimmune activation does not appear sufficient to fully explain beta cell loss in T1 D (5). Moreover, the failure of immunotherapies to cure T1 D(7) highlighted that: (i) autoimmunity may not be the sole factor involved in T1 D pathogenesis and (ii) alternative strategies that target different mechanisms of disease, such as beta cell loss, are needed in order to establish an effective treatment for T1 D. The observation that scattered beta cells are detected in individuals with long-standing T1 D(8) confirms that either new beta cells must be occurring in order to preserve the beta cell turnover (5, 9), or the destroyed beta-cells may be “different” and prone to death (10). This may suggest that the up/down-regulated expression of surface beta cell receptors may have a key role in making them visible to immune system and, more importantly, that other non-immunological determinants may modulate beta cell fate and function. Therefore, preventing the non-immunological beta cell destruction in T1D and the progressive loss of beta cells in T2D may skew the balance between beta cell generation and destruction towards the recovery of the appropriate beta cell mass, thus paving the way for novel therapeutic approaches capable of halting or delaying the very first phase of the disease. It has been shown that TMEM219, the IGFBP3 receptor, is expressed in a beta cell line and in human/murine islets, and that its ligation is toxic to beta cells. Interestingly, it has been also observed that mice transgenic for human IGFBP3 develop hyperglycemia, exhibit a reduced islets mass and show a decrease response to insulin-glucose stimulation (11), while those knocked down for IGFBP3 did not show any alteration in terms of glycometabolic control (12).

In humans, Drogan and colleagues recently published that elevated circulating levels of IGFBP3 are associated with the development of T2D (13). Moreover, a recent study by the Diabimmune Study group demonstrated that IGFBP3 levels correlate with autoantibody positivity and chance to seroconversion in children at risk for T1 D, thus suggesting a role for circulating IGFBP3 in the early development of beta cell autoimmunity (14).

TMEM219, the IGFBP3 receptor, has been already described as a death receptor, whose activation triggers Caspase8-mediated apoptosis within the target cells thus leading to their loss (4).

IGFBP3/TMEM219 Axis in Inflammatory Bowel Disease

Intestinal stem cells (ISCs) reside at the bottom of small and large intestine crypts and control the crypts regeneration and turnover. In particular, ISCs can differentiate along the crypts to generate goblet cells, enterocytes, enteroendocrine cells (4).

Inflammatory bowel disease (IBD) is an immune-mediated chronic condition that encompasses two clinical entities, Crohn's disease (CD) and ulcerative colitis (UC), and affects nearly 2.5 million of individuals in Europe and 1 million in USA (15). The pathogenesis of IBD is still under investigation, but recent evidences suggest that an impaired differentiation of ISCs towards Paneth cells, in ileal CD, and towards goblet cells in UC, may play a key-role in the onset of the disease. In particular, local signaling and inflammatory pathways in the mucosa both respond to external stimuli and preserve ISCs number and function, thus maintaining intestinal homeostasis (16). Indeed recently, Yancu et al., published results that support the role of IGFBP-3 in CD. Indeed, they demonstrated that, the knockout of IGFBP3 has a role in modulating inflammation in the Dextran-Sodium-Sulphate (DSS) colitis murine model (17).

The inventors have recently found that the insulin-like growth factor binding protein 3 (IGFBP3) receptor, namely the TMEM219 receptor, is expressed on ISCs and that its interaction with the circulating hormone IGFBP3 controls ISCs fate and function in a model of intestinal disorders in diabetes and diabetic enteropathy (4). Since diabetic enteropathy and IBD share common features, as alteration in intestinal stem cell (ISC) homeostasis and altered mucosa morphology, these results may add important insights in the still unknown IBD pathogenesis and will possibly lead to the introduction of a new therapeutic approach for IBD treatment.

Current available therapy for IBD is based on the use of anti-inflammatory and immunotherapeutic strategies, which are aggravated by several adverse effects and whose effectiveness in the long-term remains questionable. Surgery is also successfully employed in advanced state of the disease especially in UC (15). Relapsing of the disease mostly in CD is also frequent, thus highlighting the need for a different therapeutic approach. As a result, the identification of novel therapeutic targets and strategies in the treatment of IBD is of a high clinically relevance and need for the health community.

WO2016193497 and WO2016193496 (incorporated herein by reference in their entireties), describe a TMEM219 extracellular domain, ecto-TMEM, acting as an effective therapeutic agent. However, receptor constructs are less desirable as therapeutic agents than are antibodies. Therefore, there is still a need for further therapeutics agents, as antibodies or derivatives thereof, that mimic the effects of ecto-TMEM.

### SUMMARY OF THE INVENTION

Disclosed herein are antibodies that bind with high affinity and specificity to human IGF binding protein 3 (IGFBP3) and that are capable of reducing or abrogating binding of IGFBP3 to its cognate receptor, TMEM219. These neutralizing antibodies are useful in treating disorders in which IGFBP3 binding to TMEM219 contributes to the pathophysiology of the disease, including diabetic enteropathy, inflammatory bowel disease (IBD), such as ulcerative colitis and Crohn's disease, and type 1 or type 2 diabetes. Such neutralizing antibodies provide advantageous therapeutic agents that have therapeutic activities similar to the receptor-based ligand trap ecto-TMEM219.

In a first aspect, it is provided an isolated antibody or antigen binding fragment thereof that binds to human IGFBP3 with an affinity constant lower than or equal to 1.1×10⁻⁹ M and which inhibits or reduces the binding of IGFBP3 to the TMEM219 receptor.

Preferably the isolated antibody or antigen binding fragment thereof inhibits, reduces, or neutralizes the activation of the TMEM219 receptor induced by binding of IGFBP3.

Activation of the TMEM219 receptor induced by IGFBP3 may be measured by any known method in the art or as described below. In particular, IGFBP3-induced activation of a TMEM219 receptor may be measured by measuring apoptosis increase as described therein or decrease in minigut growth as known in the art and described therein and in several publications (4, 18, 27, 28).

In a preferred embodiment the isolated antibody or antigen binding fragment thereof is effective in controlling blood glucose levels in an in vivo model.

The present invention also provides an isolated antibody or antigen binding fragment thereof that has at least one activity selected from:
a—increase in IGFBP3 treated healthy subject minigut growth
b—increase in IBD-patient minigut growth;
c—increase in diabetic enteropathy serum treated healthy subject minigut growth;
d—increase in expression of EphB2 and/or LGR5 in IGFBP3 treated healthy subject minigut;
e—decrease in caspase 8 expression in IGFBP3 treated healthy subject minigut;
f—decrease in R-cell loss in IGFBP3 treated R-cell;
g—increase in expression of insulin in IGFBP3 treated R-cell; and
h—decrease in apoptosis of R-cell in IGFBP3 treated R-cell;
i—decrease in caspase 8 expression in IGFBP3 treated R-cell;
j—Decrease in insulitis score in an animal model of diabetes;
k-Decrease in diabetes onset in an animal model of diabetes.

Preferably the increase in a), b) and c) is by at least 20%; the increase in d) and e) is by at least 50%; the decrease in f) and the increase in g) is by at least 10%; the decrease in i), j) and k) is by at least 50%, preferably the decrease in k is by at least 70%.

The invention provides an isolated antibody or antigen binding fragment thereof comprising:
a. a heavy chain variable domain (VH) comprising:
   i. a CDR1 sequence of the amino acid sequence selected from the group consisting of: SEQ ID NO: 1, 4, 7 or 9;
   ii. a CDR2 sequence of the amino acid sequence selected from the group consisting of: SEQ ID NO: 2, 5, 8 or 10; and
   iii. a CDR3 sequence of the amino acid sequence selected from the group consisting of: SEQ ID NO: 3, 6 or 11; and/or
b. a light chain variable domain (VL) comprising:
   i. a CDR1 sequence of the amino acid sequence selected from the group consisting of: SEQ ID NO: 12, 15, 17, 20, 23, 25 or 27;
   ii. a CDR2 sequence of the amino acid sequence selected from the group consisting of: SEQ ID NO: 13, 18 or 21; and
   iii. a CDR3 sequence of the amino acid sequence selected from the group consisting of: SEQ ID NO: 14, 16, 19, 22, 24 or 26.

Preferably the isolated antibody or antigen binding fragment thereof comprises the CDRs as indicated in Table 2 and/or in Table 3, including Table 3.1.

Preferably it has at least one activity selected from:
a—increase in IGFBP3 treated healthy subject minigut growth
b—increase in IBD-patient minigut growth;
c—increase in diabetic enteropathy serum treated healthy subject minigut growth;
d—increase in expression of EphB2 and/or LGR5 in IGFBP3 treated healthy subject minigut;
e—decrease in caspase 8 expression in IGFBP3 treated healthy subject minigut;
f—decrease in R-cell loss in IGFBP3 treated R-cell;
g—increase in expression of insulin in IGFBP3 treated R-cell; and
h—decrease in apoptosis of R-cell in IGFBP3 treated R-cell;
i—decrease in caspase 8 expression in IGFBP3 treated R-cell.

Preferably the increase in a), b) and c) is by at least 20%; the increase in d) and e) is by at least 50%; the decrease in f) and the increase in g) is by at least 10%.

Preferably the isolated antibody or antigen binding fragment thereof comprises:
a. a heavy chain variable domain sequence of the amino acid sequence selected from the group consisting of: SEQ ID NO:28 to SEQ ID NO:36;
b. a light chain variable domain sequence of the amino acid sequence selected from the group consisting of: SEQ ID NO: 37 to SEQ ID NO:45; or
c. the light chain variable domain of (a) and the heavy chain variable domain of (b).

Preferably the isolated antibody or antigen binding fragment thereof comprises:
SEQ ID NO: 9 and SEQ ID NO: 10 and SEQ ID NO: 11 and SEQ ID NO: 27 and SEQ ID NO: 18 and SEQ ID NO: 26 or Kabat, IMGT, Chothia, AbM, or Contact CDRs of M1 or
SEQ ID NO: 4 and SEQ ID NO: 5 and SEQ ID NO: 6 and SEQ ID NO: 12 and SEQ ID NO: 13 and SEQ ID NO: 14 or Kabat, IMGT, Chothia, AbM, or Contact CDRs of E08 or
SEQ ID NO: 4 and SEQ ID NO: 5 and SEQ ID NO: 6 and SEQ ID NO: 23 and SEQ ID NO: 18 and SEQ ID NO: 24 or Kabat, IMGT, Chothia, AbM, or Contact CDRs of E20.

Preferably the isolated antibody or antigen binding fragment thereof comprises:
a. a heavy chain variable domain (VH) comprising:
   i. a CDR1 sequence of the amino acid sequence selected from the group consisting of a sequence as defined using abysis tool analysis (www.abysis.org);
   ii. a CDR2 sequence of the amino acid sequence selected from the group consisting of a sequence as defined using abysis tool analysis (www.abysis.org); and
iii. a CDR3 sequence of the amino acid sequence selected from the group consisting of a sequence as defined using abysis tool analysis (www.abysis.org); and/or
b. a light chain variable domain (VL) comprising:
   i. a CDR1 sequence of the amino acid sequence selected from the group consisting of a sequence as defined using abysis tool analysis (www.abysis.org);
   ii. a CDR2 sequence of the amino acid sequence selected from the group consisting of a sequence as defined using abysis tool analysis (www.abysis.org); and
   iii. a CDR3 sequence of the amino acid sequence selected from the group consisting of a sequence as defined using abysis tool analysis (www.abysis.org).

Still preferably the isolated antibody is E01, E02, E08, E14, E19, E20, E23, E24 or M1 or antigen binding fragment thereof, as reported in Tables 2-7.

Still preferably the isolated antibody is E01 comprising SEQ ID NO:28 and SEQ ID NO:37, E02 comprising SEQ ID NO:29 and SEQ ID NO:38, E08 comprising SEQ ID NO:30 and SEQ ID NO:39, E14 comprising SEQ ID NO:31 and SEQ ID NO:40, E19 comprising SEQ ID NO:32 and SEQ ID NO:41, E20 comprising SEQ ID NO:33 and SEQ ID NO:42, E23 comprising SEQ ID NO:34 and SEQ ID NO:43, E24 comprising SEQ ID NO:35 and SEQ ID NO:44, M1 comprising SEQ ID NO:36 and SEQ ID NO:45.

The invention also provides an isolated antibody or antigen binding fragment thereof that:

(a) binds specifically to an epitope on IGFBP3, e.g., the same or similar epitope as the epitope recognized by the monoclonal antibody E01, E02, E08, E14, E19, E20, E23, E24 or M1 comprising the sequences as defined in Tables 2-7; or
(b) cross-competes for binding with the monoclonal antibody E01, E02, E08, E14, E19, E20, E23, E24 or M1 comprising the sequences as defined in Tables 2-7; or
(c) shows the same or similar binding affinity or specificity, or both, as any of E01, E02, E08, E14, E19, E20, E23, E24 or M1 comprising the sequences as defined in Tables 2-7; or
(d) has one or more biological properties of an antibody molecule described herein, e.g., an antibody molecule chosen from, e.g., any of E01, E02, E08, E14, E19, E20, E23, E24 or M1 comprising the sequences as defined in Tables 2-7; or
(e) has one or more pharmacokinetic properties of an antibody molecule described herein, e.g., an antibody molecule chosen from, e.g., any of E01, E02, E08, E14, E19, E20, E23, E24 or M1 comprising the sequences as defined in Tables 2-7.

Preferably the isolated antibody or antigen binding fragment thereof of the invention is a human or humanized antibody.

More preferably the isolated antibody or antigen binding fragment thereof of the invention is an IgG2 or IgG4 antibody, preferably an IgG2 kappa antibody, an IgG2 lambda antibody, an IgG4 kappa antibody or an IgG4 lambda antibody, preferably said IgG2 or IgG4 is human IgG2 or human IgG4.

The invention provides an isolated polynucleotide comprising at least one sequence that encodes the antibody or antigen binding fragment thereof as defined above, preferably said polynucleotide is a cDNA.

The invention provides a vector comprising the polynucleotide as defined above, preferably said vector is selected from the group consisting of a plasmid, a viral vector, a non-episomal mammalian vector, an expression vector, and a recombinant expression vector.

The invention further provides an isolated cell comprising the polynucleotide as defined above or the vector as defined above, preferably the isolated cell is a hybridoma or a Chinese Hamster Ovary (CHO) cell or a Human Embryonic Kidney cells (HEK293).

The invention further provides the antibody or antigen binding fragment thereof or the isolated polynucleotide or the vector or the isolated cell s defined above for use as a medicament, preferably for use in the treatment of: diabetes, intestinal and/or bowel disorder, malabsorption syndrome, cachexia or diabetic enteropathy, preferably diabetes is Type I or Type II diabetes preferably the intestinal and/or bowel disorder is inflammatory bowel disease, celiac disease, ulcerative colitis, Crohn's disease or intestinal obstruction.

The invention provides also a pharmaceutical composition comprising the isolated antibody or antigen binding fragment thereof or the isolated polynucleotide or the vector or the isolated cell as defined above and pharmaceutically acceptable carrier, preferably for use in the treatment of: diabetes, intestinal and/or bowel disorder, malabsorption syndrome, cachexia or diabetic enteropathy, preferably the intestinal and/or bowel disorder is inflammatory bowel disease, celiac disease, ulcerative colitis, Crohn's disease or intestinal obstruction.

The invention provides a method of inhibiting the binding of IGFBP3 to TMEM219 receptor, comprising contacting IGFBP3 with the antibody or composition as defined above.

The invention provides a method of treatment of: diabetes, preferably Type 1 or Type 2 diabetes, intestinal and/or bowel disorder, malabsorption syndrome, cachexia or diabetic enteropathy, preferably the intestinal and/or bowel disorder is inflammatory bowel disease, IBD, celiac disease, ulcerative colitis, Crohn's disease or intestinal obstruction, the method comprising administering to a subject in need thereof a pharmaceutical composition comprising the isolated antibody or antigen binding fragment thereof or the isolated polynucleotide or the vector or the isolated cell as defined above and pharmaceutically acceptable carrier or administering to a subject in need thereof the isolated antibody or antigen binding fragment thereof or the isolated polynucleotide or the vector or the isolated cell as defined above.

The present invention also provides a method for producing an antibody or antigen binding fragment thereof, comprising obtaining the cell as defined above and producing the antibody or antigen binding fragment thereof.

In some embodiments, the combination includes an inhibitor of IGFBP3 (e.g., an anti-IGFBP3 antibody molecule as described herein). Thus, compositions and methods for detecting IGFBP3, as well as methods for treating various disorders including diabetes, as well as intestinal and/or bowel disorders, using the anti-IGFBP3 antibody molecules and combinations thereof are disclosed herein.

Accordingly, in one aspect, the invention features an antibody molecule (e.g., an isolated or recombinant antibody molecule) having one or more of the following properties:

(i) binds to IGFBP3, e.g., human IGFBP3, with high affinity, e.g., with an affinity constant of at least about 4×106 M⁻¹, preferably 107 M⁻¹, typically about 108 M⁻¹ and more typically, about 109 M⁻¹ to 10¹⁰ M⁻¹ or stronger;

(ii) inhibits or reduces binding of IGFBP3 to its receptor, TMEM;

(iii) binds specifically to an epitope on IGFBP3, e.g., a different epitope from the epitope recognized by commercial antibody LSBIO LS-C45037 or clone 83.8F9;

(iv) binds specifically to an epitope on IGFBP3, e.g., the same or similar epitope as the epitope recognized by the monoclonal antibody E01, E02, E08, E14, E19, E20, E23, E24 or M1 as defined in Tables 2-7;

(v) cross-competes for binding with the monoclonal antibody E01, E02, E08, E14, E19, E20, E23, E24 or M1 as defined in Tables 2-7;

(vi) shows the same or similar binding affinity or specificity, or both, as any of E01, E02, E08, E14, E19, E20, E23, E24 or M1 as defined in Tables 2-7;

(vii) shows the same or similar binding affinity or specificity, or both, as an antibody molecule (e.g., a heavy chain variable region and light chain variable region) described in Tables 2-7;

(viii) shows the same or similar binding affinity or specificity, or both, as an antibody molecule (e.g., a heavy chain variable region and light chain variable region) having an amino acid sequence shown in Tables 2-7;

(ix) shows the same or similar binding affinity or specificity, or both, as an antibody molecule (e.g., an heavy chain variable region and light chain variable region) encoded by the nucleotide sequence shown in Tables 6-7;

(x) binds the same or an overlapping epitope with a second antibody molecule to IGFBP3, wherein the second antibody molecule is an antibody molecule described herein, e.g., an antibody molecule chosen from E01, E02, E08, E14, E19, E20, E23, E24 or M1 as defined in Tables 2-7

(xi) has one or more biological properties of an antibody molecule described herein, e.g., an antibody molecule chosen from, e.g., any of E01, E02, E08, E14, E19, E20, E23, E24 or M1 as defined in Tables 2-7

(xii) has one or more pharmacokinetic properties of an antibody molecule described herein, e.g., an antibody molecule chosen from, e.g., any of E01, E02, E08, E14, E19, E20, E23, E24 or M1 as defined in Tables 2-7;

(xiii) inhibits one or more activities of IGFBP3, e.g., results in one or more of: an increase of at least 20% in the development of minigut from IBD-patient derived tissue sample when compared to untreated samples and/or an increase of at least 20% in the development of minigut growth in presence of IGFBP3 when compared to untreated samples or an increase of at least 20% in the development of minigut growth in presence of diabetic enteropathy serum when compared to untreated samples;

(xiv) induces an increase in EphB2 and LGR5 of at least 50% compared to the IGFBP3-treated samples; or decrease in caspase 8 expression level of at least 50% compared to the IGFBP3-treated samples; or

(xv) inhibits one or more activities of IGFBP3, e.g., results in one or more of: a reduction in beta cell loss, or an increase in Insulin; The reduction in beta cell loss or the increase in insulin is at least 10% compared to IGFBP3 treated samples;

(xvi) inhibits, reduces or neutralizes one or more activities of IGFBP3, resulting in blockade or reduction of IGFBP3 induced apoptosis;

(xvii) binds human IGFBP3 and is cross-reactive with cynomolgus IGFBP3.

Nucleic acid molecules encoding the antibody molecules, expression vectors, host cells and methods for making the antibody molecules are also provided. Immunoconjugates, multi- or bispecific antibody molecules and pharmaceutical compositions comprising the antibody molecules are also provided.

Without being bound to any theory, it is believed that IGFBP3/TMEM219 axis is dysfunctional in inflammatory bowel diseases (IBD) thus leading to ISCs loss and to altered function of the mucosal barrier, which is further invaded by microbes that trigger and sustain immune response activation and inflammation. The use of agents that block the IGFBP3-TMEM219 interaction in IBD may protect ISCs and preserve the integrity of the intestinal barrier, thus preventing the development of local inflammation. Further, activation of TMEM219 signaling increases apoptosis of beta cells through upregulation of caspase 8 expression and reduced insulin expression. IGFBP3 is increased in the serum of patients with pre-T1 D and pre-T2D as well as in newly diagnosed and long-standing diabetes patients and TMEM219 is expressed in beta cells.

An expression or overexpression of TMEM219 favors beta cells destruction and affects beta cell mass, and the consequent hyperglycemia/inflammation perpetrates the process during diabetes onset and progression. Altered glycemic control and inflammation in pre-diabetic conditions favor an increased IGFBP3 hepatic production, which may target TMEM219 expressed on pancreatic beta cells and trigger a loop where TMEM219 overexpression parallels the increase in IGFBP3 release. Then TMEM219 may trigger beta cell death and thus targeting the IGFBP3/TMEM219 axis may prevent such cell death.

It is noted that Casp8 is overexpressed in T1 D patients compare to control population. The anti-IGFBP3 antibody molecules disclosed herein can be used (alone or in combination with other agents or therapeutic modalities) to treat, prevent and/or diagnose disorders, such as diabetes, as well as intestinal and/or bowel disorders, malabsorption syndrome, inflammatory bowel disease, cachexia, IBD, celiac disease, diabetic enteropathy. Additionally, disclosed herein are methods and compositions comprising a combination of two, three or more therapeutic agents chosen from one, two, or all of the following categories (i)-(iii): (i) an agent that treat diabetes; (ii) an anti-inflammatory agent; or (iii) an immunotherapeutic agent.

The additional therapeutic agent may be selected from an agent that treat diabetes including: insulin, Insulin glargine as detailed in Vandana, 2014 (19, incorporated by reference), biguanide, glucosidase inhibitors, thiazolidinedione, DPP-4 inhibitors, GLP-1 receptor agonists as detailed in George et al 2013 (20, incorporated by reference)), an agent used to prevent diabetes, aspirin, anticoagulation and platelet anti-aggregation agents (such as enoxaparin, eparin, sulodexide); cholesterol-lowering drugs (such as statins, bile acids sequestrants, ezetimibe, fibrates as described in Marsha et al 2011 (21, incorporated by reference)); other blood pressure lowering agents (such as thiazide, ACE inhibitors, beta and alpha blockers); an anti-apoptotic agent, an anti-inflammatory agent, corticosteroids and immune suppressive agent (22, incorporated by reference), adjuvant therapy in organ transplantation, protective agent in cell therapy approach, a pain reliever, antibiotic, probiotics, TNF-alpha blockers (23, incorporated by reference), SGLT2 inhibitors (such as gliflozin derivates), integrin inhibitors (24, incorporated by reference).

Methods to measure an increase in minigut growth when compared to minigut growth in the presence of IGFBP3, and/or in the presence of diabetic enteropathy serum are known in the art and are described in several publications (4, 18, 27, 28) or as described in the method section below.

Methods to measure an increase and/or a decrease in EphB2, LGR5 or caspase 8 expression when compared to expression in the presence of IGFBP3 are known in the art and include quantitative RT-PCR, Realt-Time RT-PCR, microarray, northern blotting, RNA-Seq (29,30) or as described in the method section below.

Methods to measure a decrease in beta-cell loss when compared to beta-cell loss in the presence of IGFBP3 are known in the art and include cell proliferation assays (CFSE staining, Calcein/PI staining, Trypan Blue exclusion, BrdU staining, MTT) apoptosis assays (TUNEL, Caspase activation and detection, Annexin V binding) or as described in the method section below.

Methods to measure an increase in insulin level when compared to insulin level in the presence of IGFBP3 are known in the art and include western blots, ELISA, mass spectrometry (31-33).

Methods to measure a decrease in apoptosis when compared to apoptosis in the presence of IGFBP3 are known in the art and include DNA fragmentation, caspase activation analysis, mitochondrial membrane permeabilization, annexin V binding (34) or as described in the method section below.

In some embodiments, the antibody molecule binds to IGFBP3 with high affinity, e.g., with a KD that is about the same, or at least about 10%, 20%, 30%, 40%, 50%, 60%, 70%, 80% or 90% higher or lower than the KD of a murine anti-IGFBP3 antibody molecule or chimeric anti-IGFBP3 antibody molecule or a commercial anti-IGFBP3 antibody molecule. In some embodiments, the KD of the murine or chimeric anti-IGFBP3 antibody molecule is less than about 0.4, 0.3, 0.2, 0.1, or 0.05 nM, e.g., measured by a Biacore method or KinExA=kinetic exclusion assays. In some embodiments, the KD of the murine or chimeric anti-IGFBP3 antibody molecule is less than about 0.2 nM. In other embodiments, the KD of the murine or chimeric anti IGFBP3 antibody molecule is less than about 10, 5, 3, 2, or 1 nM, e.g., measured by binding on cells expressing IGFBP3 (e.g., 300.19 cells). In some embodiments, the KD of the murine or chimeric anti IGFBP3 antibody molecule is less than about 1 nM.

Methods to measure binding to IGFBP3 are known in the art as protein-protein interactions assays and include ELISA, co-immunoprecipitation, surface plasmon resonance, FRET-Forster resonance energy transfer (35) or as described in the method section below.

In some embodiments, the expression level of the antibody molecule is higher, e.g., at least about 0.5, 1, 2, 3, 4, 5, 6, 7, 8, 9 or 10-fold higher, than the expression level of a murine or chimeric antibody molecule, e.g., a murine, commercial or chimeric anti-IGFBP3 antibody molecule such as LSBIO LS-C45037, clone 83.8F9 or Novus NBP2-12364. In some embodiments, the antibody molecule is expressed in HEK293 cells, CHO cells or any suitable mammalian cell line known in the art.

In some embodiments, the anti-IGFBP3 antibody molecule reduces one or more IGFBP3-associated activities with an IC50 (concentration at 50% inhibition) that is about the same or lower, e.g., at least about 10%, 20%, 30%, 40%, 50%, 60%, 70%, 80% or 90% lower, than the IC50 of a murine, commercial or chimeric anti-IGFBP3 antibody molecule, e.g., a murine commercial or chimeric anti-IGFBP3 antibody molecule described herein.

In some embodiments, the anti-IGFBP3 antibody molecule has improved stability, e.g., at least about 0.5, 1, 2, 3, 4, 5, 6, 7, 8, 9 or 10-fold more stable in vivo or in vitro, than a murine, commercial or chimeric anti-IGFBP3 antibody molecule, e.g., a murine, commercial or chimeric anti-IGFBP3 antibody molecule such as LSBIO LS-C45037, clone 83.8F9 or Novus NBP2-12364.

In one embodiment, the anti IGFBP3 antibody molecule is a humanized antibody molecule.

In another embodiment, the anti-IGFBP3 antibody molecule comprises at least one antigen-binding region, e.g., a variable region or an antigen-binding fragment thereof, from an antibody described herein, e.g., an antibody chosen from any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences. In yet another embodiment, the anti-IGFBP3 antibody molecule comprises at least one, two, three or four variable regions from an antibody described herein, e.g., an antibody chosen from any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule comprises at least one or two heavy chain variable regions from an antibody described herein, e.g., an antibody chosen from any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule comprises at least one or two light chain variable regions from an antibody described herein, e.g., an antibody chosen from any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences.

In yet another embodiment, the anti-IGFBP3 antibody molecule includes a heavy chain constant region for an IgG4, e.g., a human IgG4. In one embodiment, the human IgG4 includes a substitution at position 228 (e.g., a Ser to Pro substitution). In one embodiment, the human IgG4 includes a substitution at position 235 (e.g., a Leu to Glu substitution). In one embodiment, the human IgG4 includes a substitution at position 228 (e.g., a Ser to Pro substitution) and a substitution at position 235 (e.g., a Leu to Glu substitution). In still another embodiment, anti-IGFBP3 antibody molecule includes a heavy chain constant region for an IgG1, e.g., a human IgG1. In one embodiment, the human IgG1 includes a substitution at position 297 (e.g., an Asn to Ala substitution). In one embodiment the human IgG1 includes a substitution at position 250, a substitution at position 428, or both (e.g., a Thr to Gln substitution at position 250 and/or a Met to Leu substitution at position 428). In one embodiment, the human IgG1 includes a substitution at position 234, a substitution at position 235, or both (e.g., a Leu to Ala substitution at position 234 and/or a Leu to Ala substitution at position 235). In one embodiment, the heavy chain constant region comprises an amino sequence set forth in Table 8, or a sequence substantially identical (e.g., at least 80%, 85%, 90%, 92%, 95%, 97%, 98%, 99% or higher identical) thereto.

In yet another embodiment, the anti-IGFBP3 antibody molecule includes a kappa light chain constant region, e.g., a human kappa light chain constant region. In one embodiment, the light chain constant region comprises an amino sequence set forth in Table 8, or a sequence substantially identical (e.g., at least 80%, 85%, 90%, 92%, 95%, 97%, 98%, 99% or higher identical) thereto.

In another embodiment, the anti-IGFBP3 antibody molecule includes a heavy chain constant region for an IgG4, e.g., a human IgG4, and a kappa light chain constant region, e.g., a human kappa light chain constant region, e.g., a heavy and light chain constant region comprising an amino sequence set forth in Table 8, or a sequence substantially identical (e.g., at least 80%, 85%, 90%, 92%, 95%, 97%, 98%, 99% or higher identical) thereto. In yet another embodiment, the anti-IGFBP3 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 8, or a sequence substantially identical (e.g., at least 80%, 85%, 90%, 92%, 95%, 97%, 98%, 99% or higher identical) thereto. In one embodiment, the human IgG1 or IgG4 includes a substitution at the variable region to decrease aggregation, reduce charge heterogeneity, increase affinity and modulate antigen binding; removal by mutation of instability hotspot in the CDR, putative N-glycosylation sites in the variable region as described in (26), incorporated by reference.

In another embodiment, the anti-IGFBP3 antibody molecule includes a heavy chain variable domain and a constant region, a light chain variable domain and a constant region, or both, comprising the amino acid sequence of any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences. The anti-IGFBP3 antibody molecule, optionally, comprises a leader sequence from a heavy chain, a light chain, or both.

In yet another embodiment, the anti-IGFBP3 antibody molecule includes at least one, two, or three complementarity determining regions (CDRs) from a heavy chain variable region of an antibody described herein, e.g., an antibody chosen from any of any of E01, E02, E08, E14, E19, E20, E23, E24 or M1 as defined in Tables 2-7 or a sequence substantially identical (e.g., at least 80%, 85%, 90%, 92%, 95%, 97%, 98%, 99% or higher identical) to any of the sequences present in Tables 2-7.

In yet another embodiment, the anti-IGFBP3 antibody molecule includes at least one, two, or three CDRs (or collectively all of the CDRs) from a heavy chain variable region comprising an amino acid sequence shown in Tables 2-5 or encoded by a nucleotide sequence shown in Tables 6-7. In one embodiment, one or more of the CDRs (or collectively all of the CDRs) have one, two, three, four, five, six or more changes, e.g., amino acid substitutions or deletions, relative to the amino acid sequence shown in Tables 2-5, or encoded by a nucleotide sequence shown in Tables 6-7.

In yet another embodiment, the anti-IGFBP3 antibody molecule includes at least one, two, or three CDRs from a light chain variable region of an antibody described herein, e.g., an antibody chosen from any of any of E01, E02, E08, E14, E19, E20, E23, E24 or M1 as defined in Tables 2-5 and 3.1, or encoded by the nucleotide sequence in Tables 6-7; or a sequence substantially identical (e.g., at least 80%, 85%, 90%, 92%, 95%, 97%, 98%, 99% or higher identical) to any of the aforesaid sequence.

In yet another embodiment, the anti-IGFBP3 antibody molecule includes at least one, two, or three CDRs (or collectively all of the CDRs) from a light chain variable region comprising an amino acid sequence shown in Tables 2-5 or encoded by a nucleotide sequence shown in Tables 6-7. In one embodiment, one or more of the CDRs (or collectively all of the CDRs) have one, two, three, four, five, six or more changes, e.g., amino acid substitutions or deletions, relative to the amino acid sequence shown in Tables 2-5 and 3.1, or encoded by a nucleotide sequence shown in Tables 6-7. In certain embodiments, the anti-IGFBP3 antibody molecule includes a substitution in a light chain CDR, e.g., one or more substitutions in a CDR1, CDR2 and/or CDR3 of the light chain. In another embodiment, the anti-IGFBP3 antibody molecule includes at least one, two, three, four, five or six CDRs (or collectively all of the CDRs) from a heavy and light chain variable region comprising an amino acid sequence shown in Tables 2-5, or encoded by a nucleotide sequence shown in Tables 6-7. In one embodiment, one or more of the CDRs (or collectively all of the CDRs) have one, two, three, four, five, six or more changes, e.g., amino acid substitutions or deletions, relative to the amino acid sequence shown in Tables 2-5 and 3.1 or encoded by a nucleotide sequence shown in Tables 6-7.

In one embodiment, the anti-IGFBP3 antibody molecule includes all six CDRs from an antibody described herein, e.g., an antibody chosen from any of any of E01, E02, E08, E14, E19, E20, E23, E24 or M1 as defined in Tables 2-5 and 3.1, or encoded by the nucleotide sequence in Tables 6-7, or closely related CDRs, e.g., CDRs which are identical or which have at least one amino acid alteration, but not more than two, three or four alterations (e.g., substitutions, deletions, or insertions, e.g., conservative substitutions). In one embodiment, the anti-IGFBP3 antibody molecule may include any CDR described herein. In certain embodiments, the anti-IGFBP3 antibody molecule includes a substitution in a light chain CDR, e.g., one or more substitutions in a CDR1, CDR2 and/or CDR3 of the light chain. In another embodiment, the anti-IGFBP3 antibody molecule includes at least one, two, or three CDRs according to Kabat et al. (e.g., at least one, two, or three CDRs according to the Kabat definition or other definitions as set out in Tables 2-5 and 3.1) from a heavy chain variable region of an antibody described herein, e.g., an antibody chosen from any of any of E01, E02, E08, E14, E19, E20, E23, E24 or M1 as defined in Tables 2-5 and 3.1 or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. or other definitions shown in Tables 2-5 and 3.1.

In another embodiment, the anti-IGFBP3 antibody molecule includes at least one, two, or three CDRs according to Kabat et al. (e.g., at least one, two, or three CDRs according to the Kabat or other definition as set out in Tables 2-3 and 3.1) from a light chain variable region of an antibody described herein, e.g., an antibody chosen from any of any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. or other definitions shown in Tables 2-3 and 3.1.

In yet another embodiment, the anti-IGFBP3 antibody molecule includes at least one, two, three, four, five, or six CDRs according to Kabat et al. (e.g., at least one, two, three, four, five, or six CDRs according to the Kabat or other definition as set out in Tables 2-3 and 3.1) from the heavy and light chain variable regions of an antibody described herein, e.g., an antibody chosen from any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. or other definitions shown in Tables 2-5 and 3.1.

In yet another embodiment, the anti-IGFBP3 antibody molecule includes all six CDRs according to Kabat et al. or other definition (e.g., all six CDRs according to the Kabat definition or other definition as set out in Tables 2-5 and 3.1) from the heavy and light chain variable regions of an antibody described herein, e.g., an antibody chosen from any of any of E01, E02, E08, E14, E19, E20, E23, E24 or M1 as defined in Tables 2-5, or encoded by the nucleotide sequence in Tables 6-7; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Tables 2-5 or 3.1. In one embodiment, the anti-IGFBP3 antibody molecule may include any CDR described herein.

In another embodiment, the anti-IGFBP3 antibody molecule includes at least one, two, or three Chothia or Kabat hypervariable loops (e.g., at least one, two, or three hypervariable loops according to the Chothia or Kabat definition as set out in Tables 2-5) from a heavy chain variable region of an antibody described herein, e.g., an antibody chosen from any of any of E01, E02, E08, E14, E19, E20, E23, E24 or M1 as defined in Tables 2-5, or encoded by the nucleotide sequence in Tables 6-7; or at least the amino acids from those hypervariable loops that contact IGFBP3; or which have at least one amino acid alteration, but not more than two, three or four alterations (e.g., substitutions, deletions, or insertions, e.g., conservative substitutions) relative to one, two, or three hypervariable loops according to Chothia et al. shown in Tables 2-5.

In another embodiment, the anti-IGFBP3 antibody molecule includes at least one, two, or three Chothia hypervariable loops (e.g., at least one, two, or three hypervariable loops according to the Chothia definition as set out in Tables 2-5) of a light chain variable region of an antibody described herein, e.g., an antibody chosen from any of any of E01, E02, E08, E14, E19, E20, E23, E24 or M1 as defined in Tables 2-5, including 3.1 or encoded by the nucleotide sequence in Tables 6-7; or at least the amino acids from those hypervariable loops that contact IGFBP3; or which have at least one amino acid alteration, but not more than two, three or four alterations (e.g., substitutions, deletions, or insertions, e.g., conservative substitutions) relative to one, two, or three hypervariable loops according to Chothia et al. shown in Tables 2-5, including 3.1.

In yet another embodiment, the anti-IGFBP3 antibody molecule includes at least one, two, three, four, five, or six hypervariable loops (e.g., at least one, two, three, four, five, or six hypervariable loops according to the Chothia definition as set out in Tables 2-5) from the heavy and light chain variable regions of an antibody described herein, e.g., an antibody chosen from any of any of E01, E02, E08, E14, E19, E20, E23, E24 or M1 as defined in Tables 2-5, including 3.1; or as described in Tables 2-5, including 3.1, or encoded by the nucleotide sequence in Tables 6-7; or at least the amino acids from those hypervariable loops that contact IGFBP3; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Tables 2-5, including 3.1.

In one embodiment, the anti-IGFBP3 antibody molecule includes all six hypervariable loops (e.g., all six hypervariable loops according to the Chothia definition as set out in Tables 2-5) of an antibody described herein, e.g., an antibody chosen from any of any of E01, E02, E08, E14, E19, E20, E23, E24 or M1 as defined in Tables 2-5, including 3.1 or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Tables 2-5. In one embodiment, the anti-IGFBP3 antibody molecule may include any hypervariable loop described herein.

In still another embodiment, the anti-IGFBP3 antibody molecule includes at least one, two, or three hypervariable loops that have the same canonical structures as the corresponding hypervariable loop of an antibody described herein, e.g., an antibody chosen from any of any of E01, E02, E08, E14, E19, E20, E23, E24 or M1 as defined in Tables 2-5, including 3.1, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In certain embodiments, the anti-IGFBP3 antibody molecule includes a combination of CDRs or hypervariable loops defined according to the Kabat et al. and Chothia et al. or any other definition known in the art.

In one embodiment, the anti-IGFBP3 antibody molecule includes at least one, two or three CDRs or hypervariable loops from a heavy chain variable region of an antibody described herein, e.g., an antibody chosen from any of any of E01, E02, E08, E14, E19, E20, E23, E24 or M1 as defined in Tables 2-5, including 3.1, according to the Kabat and Chothia or other definition (e.g., at least one, two, or three CDRs or hypervariable loops according to the Kabat and Chothia or other definition as set out in Tables 2-5, including 3.1); or encoded by the nucleotide sequence in Tables 6-7; or a sequence substantially identical (e.g., at least 80%, 85%, 90%, 92%, 95%, 97%, 98%, 99% or higher identical) to any of the aforesaid sequences, or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia or other definitions shown in Tables 2-5, including 3.1.

For example, the anti-IGFBP3 antibody molecule can include VH CDR1 according to Kabat et al. or VH hypervariable loop 1 according to Chothia et al., or a combination thereof, e.g., as shown in Tables 2-5, including 3.1. The anti-IGFBP3 antibody molecule can further include, e.g., VH CDRs 2-3 according to Kabat et al. and VL CDRs 1-3 according to Kabat et al., e.g. or other definitions as shown in Tables 2-5, including 3.1. Accordingly, in some embodiments, framework regions are defined based on a combination of CDRs defined according to Kabat et al. and hypervariable loops defined according to Chothia et al. For example, the anti-IGFBP3 antibody molecule can include VH FR1 defined based on VH hypervariable loop 1 according to Chothia et al. and VH FR2 defined based on VH CDRs 1-2 according to Kabat et al., e.g., or other definitions as shown in Tables 2-5, including 3.1. The anti-IGFBP3 antibody molecule can further include, e.g., VH FRs 3-4 defined based on VH CDRs 2-3 according to Kabat et al. or other definitions and VL FRs 1-4 defined based on VL CDRs 1-3 according to Kabat et al. or other definitions.

The anti-IGFBP3 antibody molecule can contain any combination of CDRs or hypervariable loops according to the Kabat and Chothia definitions. In one embodiment, the anti-IGFBP3 antibody molecule includes at least one, two or three CDRs from a light chain variable region of an antibody described herein, e.g., an antibody chosen from any of any of E01, E02, E08, E14, E19, E20, E23, E24 or M1 as defined in Tables 2-5, including 3.1 according to the Kabat and Chothia or other definitions (e.g., at least one, two, or three CDRs according to the Kabat and Chothia definition as set out in Tables 2-5). Preferred anti-IGFBP3 antibodies are E01, E02, E08, E14, E19, E20, E23, E24 or M1 as defined in Tables 2-5, including 3.1.

In an embodiment, e.g., an embodiment comprising a variable region, a CDR (e.g., Chothia CDR or Kabat CDR), or other sequence referred to herein, e.g., in Tables 2-5, including 3.1, the antibody molecule is a monospecific antibody molecule, a bispecific antibody molecule, or is an antibody molecule that comprises an antigen binding fragment of an antibody, e.g., a half antibody or antigen binding fragment of a half antibody. In embodiments the antibody molecule is a bispecific antibody molecule having a first binding specificity for IGFBP3 and a second binding specificity for TNF-alpha, integrin, IL1, IL12 and IL23, CD3, CD20, CD80, CD86.

In one embodiment, the anti-IGFBP3 antibody molecule includes:

(i) a heavy chain variable region (VH) including a VHCDR1 amino acid sequence chosen from any one of SEQ ID NO: 1, 4, 7 or 9; a VHCDR2 amino acid sequence chosen from any one of SEQ ID NO: 2, 5, 8 or 10; and a VHCDR3 amino acid sequence chosen from any one of SEQ ID NO: 3, 6 or 11; and/or

(ii) a light chain variable region (VL) including a VLCDR1 amino acid sequence chosen from any one of SEQ ID NO: 12, 15, 17, 20, 23, 25 or 27, a VLCDR2 amino acid sequence chosen from any one of SEQ ID NO: 13, 18 or 21, and a VLCDR3 amino acid sequence chosen from SEQ ID NO: 14, 16, 19, 22, 24 or 26.

In another embodiment, the anti-IGFBP3 antibody molecule includes:

(i) a heavy chain variable region (VH) including a VHCDR1 amino acid sequence chosen from SEQ ID NO: 1, SEQ ID NO: 4, SEQ ID NO: 7 or SEQ ID NO:9; a VHCDR2 amino acid sequence of SEQ ID NO: 2, SEQ ID NO: 5, SEQ ID NO: 8 or SEQ ID NO: 10 and a VHCDR3 amino acid sequence of SEQ ID NO: 3, SEQ ID NO: 6 or SEQ ID NO: 11 and

(ii) a light chain variable region (VL) including a VLCDR1 amino acid sequence of SEQ ID NO: 12, SEQ ID NO: 15, SEQ ID NO: 17, SEQ ID NO: 20, SEQ ID NO: 23, SEQ ID NO: 25 or SEQ ID NO: 27, a VLCDR2 amino acid sequence of SEQ ID NO: 13, SEQ ID NO: 18 or SEQ ID NO: 21, and a VLCDR3 amino acid sequence of SEQ ID NO: 14, SEQ ID NO: 16, SEQ ID NO: 19, SEQ ID NO: 22, SEQ ID NO: 24 or SEQ ID NO: 26. In one embodiment, the light or the heavy chain variable framework (e.g., the region encompassing at least FR1, FR2, FR3, and optionally FR4) of the anti-IGFBP3 antibody molecule can be chosen from: (a) a light or heavy chain variable framework including at least 80%, 85%, 87% 90%, 92%, 93%, 95%, 97%, 98%, or preferably 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In certain embodiments, the anti-IGFBP3 antibody molecule comprises a heavy chain variable domain having at least one, two, three, four, five, six, seven, ten, fifteen, twenty or more changes, e.g., amino acid substitutions or deletions.

In one embodiment, the heavy or light chain variable region, or both, of the anti-IGFBP3 antibody molecule includes an amino acid sequence encoded by a nucleic acid sequence described herein or a nucleic acid that hybridizes to a nucleic acid sequence described herein (e.g., a nucleic acid sequence as shown in Tables 6 and 7) or its complement, e.g., under low stringency, medium stringency, or high stringency, or other hybridization condition described herein.

In another embodiment, the anti-IGFBP3 antibody molecule comprises at least one, two, three, or four antigen-binding regions, e.g., variable regions, having an amino acid sequence as set forth in Tables 2-5, or a sequence substantially identical thereto (e.g., a sequence at least about 85%, 90%, 95%, 99% or more identical thereto, or which differs by no more than 1, 2, 5, 10, or 15 amino acid residues from the sequences shown in Tables 2-5. In another embodiment, the anti-IGFBP3 antibody molecule includes a VH and/or VL domain encoded by a nucleic acid having a nucleotide sequence as set forth in Tables 6-7, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 6-7.

In yet another embodiment, the anti-IGFBP3 antibody molecule comprises at least one, two, or three CDRs from a heavy chain variable region having an amino acid sequence as set forth in Tables 2-5, including 3.1, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-IGFBP3 antibody molecule comprises at least one, two, or three CDRs from a light chain variable region having an amino acid sequence as set forth in Tables 2-5, including 3.1, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-IGFBP3 antibody molecule comprises at least one, two, three, four, five or six CDRs from heavy and light chain variable regions having an amino acid sequence as set forth in Tables 2-5, including 3.1, or a sequence substantially homologous thereto (e.g., a sequence at least about 85%, 90%, 95%, 99% or more identical thereto, and/or having one, two, three or more substitutions, insertions or deletions, e.g., conserved substitutions).

In yet other embodiments, the anti-IGFBP3 antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG4 (e.g., human IgG1, IgG2 or IgG4). In one embodiment, the heavy chain constant region is human IgG1. In another embodiment, the anti-IGFBP3 antibody molecule has a light chain constant region chosen from, e.g., the light chain constant regions of kappa or lambda. In one embodiment, the constant region is altered, e.g., mutated, to modify the properties of the anti-IGFBP3 antibody molecule (e.g., to increase or decrease one or more of: Fc receptor binding, antibody glycosylation, the number of cysteine residues, effector cell function, complement function, half-life, aggregation and stability). In certain embodiments, the anti-IGFBP3 antibody molecules comprises a human IgG4 mutated

In one embodiment, the anti-IGFBP3 antibody molecule is isolated or recombinant.

In one embodiment, the anti-IGFBP3 antibody molecule is a humanized or human antibody molecule.

The invention also features a nucleic acid molecule that comprise one or both nucleotide sequences that encode heavy and light chain variable regions, CDRs, hypervariable loops, framework regions of the anti-IGFBP3 antibody molecules, as described herein. In certain embodiments, the nucleotide sequence that encodes the anti-IGFBP3 antibody molecule is codon optimized. For example, the invention features a first and second nucleic acid encoding heavy and light chain variable regions, respectively, of an anti-IGFBP3 antibody molecule chosen from one or more of, e.g., any of E01, E02, E08, E14, E19, E20, E23, E24 or M1 as defined in Tables 2-5, including 3.1, or a sequence substantially identical thereto. For example, the nucleic acid can comprise a nucleotide sequence as set forth in Tables 6-7, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 6-7).

In other embodiments, the nucleic acid molecule comprises a nucleotide sequence that encodes a heavy chain variable domain and/or a heavy chain constant region comprising the amino acid sequence of any of E01, E02, E08, E14, E19, E20, E23, E24 or M1 as defined in Tables 2-5, including 3.1; or the nucleotide sequence in Tables 6-7; or a sequence substantially identical (e.g., a sequence at least about 85%, 90%, 95%, 99% or more identical) to any of the aforesaid sequences.

In other embodiments, the nucleic acid molecule comprises a nucleotide sequence that encodes a light chain variable domain and/or a light chain constant region comprising the amino acid sequence of any of E01, E02, E08, E14, E19, E20, E23, E24 or M1 as defined in Tables 2-5, including 3.1; or the nucleotide sequence in Tables 6-7; or a sequence substantially identical (e.g., a sequence at least about 85%, 90%, 95%, 99% or more identical) to any of the aforesaid sequences.

The aforesaid nucleotide sequences encoding the anti-IGFBP3 heavy and light chain variable domain and constant regions can be present in a separate nucleic acid molecule, or in the same nucleic acid molecule. In certain embodiments, the nucleic acid molecules comprise a nucleotide sequence encoding a leader sequence.

In certain embodiments, the nucleic acid molecule comprises a nucleotide sequence encoding at least one, two, or three CDRs, or hypervariable loops, from a heavy chain variable region having an amino acid sequence as set forth in Tables 2-5, or a sequence substantially homologous thereto (e.g., a sequence at least about 85%, 90%, 95%, 99% or more identical thereto, and/or having one, two, three or more substitutions, insertions or deletions, e.g., conserved substitutions).

In another embodiment, the nucleic acid molecule comprises a nucleotide sequence encoding at least one, two, or three CDRs, or hypervariable loops, from a light chain variable region having an amino acid sequence as set forth in Tables 6-7, or a sequence substantially homologous thereto (e.g., a sequence at least about 85%, 90%, 95%, 99% or more identical thereto, and/or having one, two, three or more substitutions, insertions or deletions, e.g., conserved substitutions).

In yet another embodiment, the nucleic acid molecule comprises a nucleotide sequence encoding at least one, two, three, four, five, or six CDRs, or hypervariable loops, from heavy and light chain variable regions having an amino acid sequence as set forth in Tables 2-5, including 3.1, or a sequence substantially homologous thereto (e.g., a sequence at least about 85%, 90%, 95%, 99% or more identical thereto, and/or having one, two, three or more substitutions, insertions or deletions, e.g., conserved substitutions).

In another embodiment, the nucleic acid molecule includes one or more heavy chain framework region (e.g., any of VHFW1 (type a), VHFW1 (type b), VHFW1 (type c), VHFW1 (type d), VHFW2 (type a), VHFW2 (type a′), VHFW2 (type b), VHFW2 (type c), VHFW2 (type d), VHFW2 (type e), VHFW3 (type a), VHFW3 (type b), VHFW3 (type c), VHFW3 (type d), VHFW3 (type e), or VHFW4, or any combination thereof, e.g., a framework combination as described herein) for any of E01, E02, E08, E14, E19, E20, E23, E24 or M1 as defined in Tables 2-5, including 3.1, or a sequence substantially identical thereto. For example, the nucleic acid molecule can comprise a nucleotide sequence as set forth in Tables 2-5, including 3.1, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 2-5, including 3.1).

In another embodiment, the nucleic acid molecule includes one or more light chain framework region (e.g., any of VLFW1 (type a), VLFW1 (type b), VLFW1 (type c), VLFW1 (type d), VLFW1 (type e), VLFW1 (type f), VLFW2 (type a), VLFW2 (type c), VLFW3 (type a), VLFW3 (type b), VLFW3 (type c), VLFW3 (type d), VLFW3 (type e), VLFW3 (type f), VLFW3 (type g), or VLFW4, or any combination thereof, e.g., a framework combination as described herein) for of any of E01, E02, E08, E14, E19, E20, E23, E24 or M1 as defined in Tables 2-5, including 3.1, or a sequence substantially identical thereto. For example, the nucleic acid molecule can comprise a nucleotide sequence as set forth in Tables 6-7, or a sequence substantially identical thereto (e.g., a sequence at least about 85%, 90%, 95%, 99% or more identical thereto, or which differs by no more than 3, 6, 15, 30, or 45 nucleotides from the sequences shown in Tables 6-7).

In another embodiment, the nucleic acid molecule includes one or more heavy chain framework region and one or more light chain framework region as described herein.

The heavy and light chain framework regions may be present in the same vector or separate vectors.

In another aspect, the application features host cells and vectors containing the nucleic acids described herein or modified for codon optimization according to known methods. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell.

In one aspect, the invention features a method of providing an antibody molecule described herein. The method includes: providing a IGFBP3 antigen (e.g., an antigen comprising at least a portion of a IGFBP3 epitope); obtaining an antibody molecule that specifically binds to the IGFBP3 polypeptide; and evaluating if the antibody molecule specifically binds to the IGFBP3 polypeptide, or evaluating efficacy of the antibody molecule in modulating, e.g., inhibiting, the activity of the IGFBP3. The method can further include administering the antibody molecule to a subject, e.g., a human or non-human animal.

In another aspect, the invention provides, compositions, e.g., pharmaceutical compositions, which include a pharmaceutically acceptable carrier, excipient or stabilizer, and at least one of the anti-IGFBP3 antibody molecules described herein. In one embodiment, the composition, e.g., the pharmaceutical composition, includes a combination of the antibody molecule and one or more agents, e.g., a therapeutic agent or other antibody molecule, as described herein. In one embodiment, the antibody molecule is conjugated to a label or a therapeutic agent.

The anti-IGFBP3 antibody molecules disclosed herein can inhibit, reduce or neutralize one or more activities of IGFBP3 as indicated above. Thus, such antibody molecules can be used to treat or prevent disorders where the inhibition, reduction or neutralization of IGFBP3-induced activities in a subject is desired.

Uses of the Anti-IGFBP3 Antibody Molecules

The present antibodies are used in methods of treatment of various disorders or conditions such as diabetes, as well as intestinal bowel diseases, malabsorption syndrome, inflammatory bowel disease, cachexia, Crohn's disease, ulcerative colitis, celiac disease, diabetic enteropathy.

Accordingly, in another aspect, a method of modulating the IGFBP3/TMEM219 axis in a subject is provided. The method comprises administering to the subject an anti-IGFBP3 antibody molecule disclosed herein (e.g., a therapeutically effective amount of an anti-IGFBP3 antibody molecule), alone or in combination with one or more agents or procedures, such that the IGFBP3/TMEM219 axis in the subject is modulated. In one embodiment, the antibody molecule inhibits, reduce or neutralize or block the IGFBP3/TMEM219 axis activity in the subject. The subject can be a mammal, e.g., a primate, preferably a higher primate, e.g., a human (e.g., a patient having, or at risk of having, a disorder described herein). In one embodiment, the subject is in need of inhibiting, reducing, neutralizing or blocking the IGFBP3/TMEM219 axis. In one embodiment, the subject has, or is at risk of, having a disorder described herein, e.g, diabetes, or inflammatory bowel disorder (IBD), malabsorption syndrome, irritable bowel disease, cachexia, celiac disease, diabetic enteropathy as described herein.

### BRIEF DESCRIPTION OF THE DRAWINGS

FIG. 1. IGFBP3-Ecto-TMEM219 binding in the presence of newly generated anti-IGFBP3 mAbs (10 μg/mL) or Ecto-TMEM219 (10 μg/mL) alone tested by using a competitive ELISA screening assay. In this assay, the microtiter plate was coated with rhIGFBP3, and labeled ecto-TMEM219 added. The monoclonal antibody M1 was added and its ability to displace ecto-TMEM219 was assessed by measuring absorbance after the plate was washed. Newly generated anti-IGFBP3 antibody M1 achieves a high reduction in Ecto-TMEM219 signal (1-way ANOVA, **** p<0.0001).

FIG. 2. Effects of anti-IGFBP3 mAbs in rescuing human mini-gut growth upon IGFBP3 exposure (50 ng/mL). Mini-guts were generated from crypts obtained from human healthy control. The newly generated anti-IGFBP3 mAb M1 (10 μg/mL) and Ecto-TMEM219 (130 ng/mL) were tested on mini-guts treated with IGFBP3. Self-renewal properties were assessed by morphology evaluation. Development of large crypts organoids with at least one crypt domain was considered as main criteria. Mini-guts development was rescued by the anti-IGFBP3 mAb tested. **** p<0.001 vs. hIGFBP3.

FIG. 3. ISCs (intestinal stem cells) markers expression is re-established by newly generated anti-IGFBP3 mAb in IGFBP3-treated mini-gut. Normalized mRNA expression of ISCs markers EphB2 (A) and LGR5 (B) analyzed by using RT-PCR in mini-guts cultured with IGFBP3 and selected anti-IGFBP3 mAbs/Ecto-TMEM219. * p<0.05 vs. IGFBP3.

FIG. 4. Caspase 8 expression is down-regulated by newly generated anti-IGFBP3 mAb in IGFBP3-treated mini-guts. Normalized mRNA expression of Caspase 8 analyzed by using RT-PCR in mini-guts cultured with IGFBP3 (50 ng/mL) and selected anti-IGFBP3 mAbs (10 μg/mL). **** p<0.001 vs. IGFBP3.

FIG. 5. Effects of anti-IGFBP3 mAbs in rescuing mini-guts growth in IBD re-challenged with IGFBP3 (50 ng/mL). Mini-guts were generated from crypts obtained from patients with Crohn's disease (CD) and re-challenged with/without IGFBP3 (50 ng/mL) and newly generated anti-IGFBP3 mAb M1 (10 μg/mL) or Ecto-TMEM219 (130 ng/mL). Self-renewal properties were assessed by morphology evaluation. Development of large crypts organoids with at least one crypt domain was considered as main criteria. Mini-guts development was rescued by the anti-IGFBP3 mAb tested. *p<0.05, *** p<0.01 vs. hIGFBP3 or vs. CD.

FIG. 6. Effects of anti-IGFBP3 mAbs in rescuing murine mini-guts growth upon IGFBP3 exposure (50 ng/mL). Mini-guts were generated from crypts obtained from control mice C57BL6/J. The newly generated anti-IGFBP3 mAb M1 (10 μg/mL) and Ecto-TMEM219 (130 ng/mL) were tested on mini-guts treated with IGFBP3. Self-renewal properties were assessed by morphology evaluation. Development of large crypts organoids with at least one crypt domain was considered as main criteria. Mini-guts development was rescued by the anti-IGFBP3 mAb tested. **** p<0.01 vs. mIGFBP3.

FIG. 7. Caspase 8 expression is down-regulated by newly generated anti-IGFBP3 mAb in IGFBP3-treated human beta-cell line. Normalized mRNA expression of Caspase 8 analyzed by using RT-PCR in beta-cells cultured with IGFBP3 (50 ng/mL) and selected anti-IGFBP3 mAb (10 μg/mL). ** p<0.01 vs. IGFBP3.

FIG. 8. Effects of anti-IGFBP3 mAbs in rescuing human mini-gut growth upon IGFBP3 exposure (50 ng/mL). Mini-guts were generated from crypts obtained from human healthy control. The newly generated anti-IGFBP3 mAbs (10 μg/mL) and Ecto-TMEM219 (130 ng/mL) were tested on mini-guts treated with IGFBP3. Self-renewal properties were assessed by morphology evaluation. Development of large crypts organoids with at least one crypt domain was considered as main criteria. Mini-guts development was rescued by the anti-IGFBP3 mAb tested. *** p<0.01, **** p<0.001 vs. hIGFBP3.

FIG. 9. ISCs (intestinal stem cells) markers expression is re-established by newly generated anti-IGFBP3 mAbs in IGFBP3-treated mini-gut. Normalized mRNA expression of ISCs markers EphB2 (A) and LGR5 (B) analyzed by using RT-PCR in mini-guts cultured with IGFBP3 and selected anti-IGFBP3 mAbs/Ecto-TMEM219. * p<0.05 vs. IGFBP3.

FIG. 10. Caspase 8 expression is down-regulated by newly generated anti-IGFBP3 mAbs in IGFBP3-treated mini-guts. Normalized mRNA expression of Caspase 8 analyzed by using RT-PCR in mini-guts cultured with IGFBP3 (50 ng/mL) and selected anti-IGFBP3 mAbs (10 μg/mL). **** p<0.001 vs. IGFBP3.

FIG. 11. Effects of anti-IGFBP3 mAbs in rescuing murine mini-guts growth upon IGFBP3 exposure (50 ng/mL). Mini-guts were generated from crypts obtained from control mice C57BL6/J. The newly generated anti-IGFBP3 mAbs (10 μg/mL) and Ecto-TMEM219 (130 ng/mL) were tested on mini-guts treated with IGFBP3. Self-renewal properties were assessed by morphology evaluation. Development of large crypts organoids with at least one crypt domain was considered as main criteria. Mini-guts development was rescued by the anti-IGFBP3 mAb tested. ** p<0.01, *** p<0.01 vs. mIGFBP3.

FIG. 12. Caspase 8 expression is down-regulated by newly generated anti-IGFBP3 mAb in human beta-cell line exposed to pooled T1D serum. Normalized mRNA expression of Caspase 8 analyzed by using RT-PCR in beta-cells cultured with pooled T1D serum and selected anti-IGFBP3 mAb (10 μg/mL). *** p<0.001 vs. IGFBP3.

FIG. 13. Experimental timelines

FIG. 14. Effect of newly generated anti-IGFBP3 mAbs on diabetes onset in T1 D mice model (A) Anti-IGFBP3 mAbs effect in preventing diabetes onset in NOD mice at 24 weeks of age and (B) in preserving blood glucose levels. Anti-IGFBP3 mAbs prevented diabetes onset in 80% of mice. Diabetes-free are the normoglycemic mice.

Blood glucose >250 mg/dl for three consecutive measurements defined diabetes onset. Diabetes-free mice do not have Blood glucose >250 mg/dl for three consecutive measurements.

FIG. 15. Serial paraffin sections of pancreatic tissue obtained at euthanasia were prepared, stained with H&E and islet morphology was analyzed microscopically. (A-i) Representative images are shown; original magnification 20×. (A-li) Representative images of insulin staining (brown color) are shown; original magnification 20×. (B) Insulitis scores are shown. In (B), the extent of cell infiltration was scored from 0 through 4. Insulitis was scored by examining a minimum of 30 islets per animal.

### DETAILED DESCRIPTION OF THE INVENTION

The antibodies of the invention specifically bind human IGFBP3. As discussed herein, the antibodies of the invention are collectively referred to as “anti-IGFBP3 antibodies”. All such antibodies are encompassed by the discussion herein. The respective antibodies can be used alone or in combination in the methods of the invention.

By “antibodies that specifically bind” IGFBP3 is intended that the antibodies will not substantially cross react with another, non-homologous, human polypeptide. By “not substantially cross react” is intended that the antibody or fragment has a binding affinity for a non-homologous protein which is less than 10%, more preferably less than 5%, and even more preferably less than 1%, of the binding affinity for IGFBP3.

In various embodiments, an antibody that “specifically binds” IGFBP3, as used herein, includes antibodies that bind human IGFBP3 with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured with an Octet biolayer interferometry device or in a surface plasmon resonance assay, for example using the BIAcore™ system (Biacore Life Sciences division of GE Healthcare, Piscataway, N.J.) or kinetic exclusion assays or any known method in the art.

The term “antibody” herein is used in the broadest sense understood in the art, including all polypeptides described as antibodies in (25), incorporated herein by reference.

For example, the term “antibody”, as used herein encompasses monoclonal antibodies, polyclonal antibodies, monospecific and multispecific antibodies (e.g., bispecific antibodies), and antibody fragments so long as the fragment exhibits the desired antigen-binding activity (antigen-binding fragments). The term has its broadest art-recognized meaning and includes all known formats, including, without limitation: bivalent monospecific monoclonal antibodies, bivalent bispecific antibodies, trivalent trispecific antibodies, F(ab) fragments, F(ab)′2 fragments, scFv fragments, diabodies, single domain antibodies, including camelid VHH single domain antibodies, tandabs, and flexibodies.

The terms “antigen-binding fragment” of an antibody or equivalently “antigen-binding portion” of an antibody and the like, as used herein, include any naturally occurring, enzymatically obtainable, synthetic, or genetically engineered polypeptide or glycoprotein that comprises a portion of an antibody and that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen.

In particular embodiments, an antigen-binding fragment of an antibody comprises at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

The term “antigen-binding fragment” of an antibody further includes single domain antibodies.

A single-domain antibody is an antibody fragment consisting of a single monomeric variable antibody domain. In some embodiments, the single-domain antibody is derived from the variable domain of the antibody heavy chain from camelids (also termed nanobodies, or VHH fragments). In some embodiments, the single-domain antibody is an autonomous human heavy chain variable domain (aVH) or VNAR fragments derived from sharks.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

The antibody or binding molecule of the invention can further be linked to an active substance, preferably a nanoparticle or a radionucleotide.

As used herein, the term “antigen binding molecule” refers in its broadest sense to a molecule that specifically binds an antigenic determinant. Examples of antigen binding molecules are antibodies, including antigen-binding antibody fragments, and scaffold antigen binding proteins.

The term “antigen binding moiety” refers to the portion of an antigen binding molecule that specifically binds to an antigenic determinant. Antigen binding moieties include antibodies and antigen-binding fragments thereof, such as scFv, that are capable of specific binding to an antigen on a target cell. In a particular aspect, the antigen binding moiety is able to direct the entity to which it is attached, such as a cell, to a target site. In addition, antigen binding moieties capable of specific binding to a target cell antigen include scaffold antigen binding proteins as defined herein below, e.g. binding domains which are based on designed repeat proteins or designed repeat domains such as designed ankyrin repeat proteins (DARPins) (see e.g. WO 2002/020565) or Lipocalins (Anticalin).

Designed Ankyrin Repeat Proteins (DARPins), which are derived from Ankyrin, which is a family of proteins that mediate attachment of integral membrane proteins to the cytoskeleton. A single ankyrin repeat is a 33-residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028.

In certain embodiments, antibodies and antigen binding molecules provided herein are altered to increase or decrease the extent to which the antigen binding moiety is glycosylated. Glycosylation variants of the molecules may be conveniently obtained by altering the amino acid sequence such that one or more glycosylation sites is created or removed. Where the antigen binding molecule comprises an Fc region, the carbohydrate attached thereto may be altered. In one aspect, variants of antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of antigen binding molecules of the invention include those with bisected oligosaccharides, e.g., in which a biantennary oligosaccharide attached to the Fc region is bisected by GIcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.) and WO 1999/22764 (Raju, S.).

In certain embodiments, it may be desirable to create cysteine engineered variants of the antibody or antigen binding molecule of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the antibody or antigen binding molecules provided herein may be further modified to contain additional non-proteinaceous moieties that are known in the art and readily available. The moieties suitable for derivatization of the antibody or antigen binding molecule include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof.

Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed. In another aspect, immunoconjugates of the antigen binding molecules provided herein may be obtained. An “immunoconjugate” is an antibody conjugated to one or more heterologous molecule(s), including but not limited to a cytotoxic agent.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity. In certain embodiments, the constant region is an IgG1, IgG2, IgG3, IgG4 constant region.

The invention encompasses in various embodiments antibodies having one or more mutations in the hinge, CH2 or CH3 region which may be desirable, for example, in production, to improve the yield of the desired antibody form. In some embodiments, for example, the antibodies described herein comprise a human IgG4 constant region. In particular embodiments, the IgG4 constant region has a single amino acid substitution in the hinge region of the human IgG4 hinge which reduced Fab arm exchange (Angal et al. (1993) Molecular Immunology 30:105) to levels typically observed using a human IgG1 hinge.

In certain embodiments, the antibody comprises one or more mutations in the constant region that increase serum half-life, including those described in U.S. Pat. Nos. 7,083,784, 8,323,962 and Dall'Aqua et al., J. Biol. Chem. 281(33):23514-23524 (2006); Hinton et al., J. Immunology 176:346-356 (2006); Yeung et al., J. Immunology 182:7663-7671 (2009); and Petkova et al., Intn'l Immunology, 18: 1759-1769 (2006), incorporated herein by reference in their entireties.

The term “human antibody”, as used herein, is intended to include antibodies having variable and constant regions derived from human germline immunoglobulin sequences. The human antibodies featured in the invention may in various embodiments nonetheless include amino acid residues not encoded by human germline immunoglobulin sequences (e.g., mutations introduced by random or site-specific mutagenesis in vitro or by somatic mutation in vivo), for example in the CDRs and in in some embodiments CDR3. However, the term “human antibody”, as used herein, is not intended to include antibodies in which CDR sequences are derived from the germline of another mammalian species, such as a mouse, which have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295, incorporated herein by reference in its entirety) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-IGFBP3 antibodies described herein and useful for the methods featured herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases.

The present invention includes in various embodiments antibodies and methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”).

Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes anti-IGFBP3 antibodies and methods involving the use of anti-IGFBP3 antibodies comprising variants of any of the HCVR, LCVR, and/or CDR amino acid sequences disclosed herein having one or more conservative substitutions. For example, the present invention includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IGFBP3 antibody are bioequivalent if they are pharmaceutically equivalent, meaning they contain the same amount of active ingredient (e.g., IGFBP3 antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

The invention in certain embodiments relates to antibodies and methods comprising administering to the subject an antibody which comprises the heavy chain variable region comprising a sequence chosen from the group of: SEQ ID NO:28 to SEQ ID NO:36 and the light chain variable region comprising a sequence chosen from the group of: SEQ ID NO:37 to SEQ ID NO:45. The disclosure provides pharmaceutical compositions comprising such antibody, and methods of using these compositions.

The antibody is administered to the subject in various embodiments in a formulation comprising suitable carriers, excipients, and other agents to provide improved transfer, delivery, tolerance, and the like, and suitable for an intravenous or subcutaneous injection.

The injectable preparations may be prepared by methods publicly known. For example, injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 20 or 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injectable preparation thus prepared can be filled in an appropriate ampoule.

The antibody according to the invention can be administered to the subject using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer an antibody (or pharmaceutical formulation comprising the antibody). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen and/or autoinjector delivery devices having applications in subcutaneous delivery of a pharmaceutical composition of the present invention include, but are not limited to, the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), the HUMIRA™ Pen (Abbott Labs, Abbott Park, Ill.), the DAI® Auto Injector (SHL Group) and any auto-injector featuring the PUSHCLICK™ technology (SHL Group), to name only a few.

In one embodiment, the antibody is administered with a prefilled syringe. In another embodiment, the antibody is administered with a prefilled syringe containing a safety system. For example, the safety system prevents an accidental needlestick injury. In various embodiments, the antibody is administered with a prefilled syringe containing an ÈRIS™ safety system (West Pharmaceutical Services Inc.). See also U.S. Pat. Nos. 5,215,534 and 9,248,242, incorporated herein by reference in their entireties. In another embodiment, the antibody is administered with an auto-injector. In various embodiments, the antibody is administered with an auto-injector featuring the PUSHCLICK™ technology (SHL Group). In various embodiments, the auto-injector is a device comprising a syringe that allows for administration of a dose of the composition and/or antibody to a subject. See also U.S. Pat. Nos. 9,427,531 and 9,566,395, incorporated herein by reference in their entireties.

According to the invention, “subject” means a human subject or human patient.

### EXAMPLES

Methods

Patients and Study Design

Healthy control subjects were individuals lacking a diagnosis of inflammatory bowel disease (IBD) (CTRL) and were enrolled from patients undergoing colonoscopy or intestinal surgery for diverticulosis, colon cancer, irritable bowel syndrome.

CD individuals had a long history of Crohn's disease and were enrolled at the moment of surgery procedure for disease complications (strictures, fistulas) or during an endoscopy routine examination before undergoing surgery. All subjects provided informed consent before study enrollment.

Animal Studies

C57BL/6J (B6) mice were obtained from Charles River Italian Laboratories (Calco, Italy) and were cared for and used in accordance with the Italian law on animal care N° 116/1992 and the European Communities Council Directive EEC/609/86.

Recombinant Proteins and Interventional Studies

Recombinant human IGFBP3 was obtained from Life Technologies (IGFBP3, Life Technologies, 10430H07H5). Ecto-TMEM219, which is the extracellular domain of the TMEM219 receptor was used as a positive control. Ecto-TMEM219 has been shown to successfully prevent IGFBP3-mediated injury in vitro and vivo, in relevant disease models. See WO 2016/193496 and WO 2016/193497. Ecto-TMEM was obtained through Genescript's customized protein service. The protein, produced in E. coli, has the following amino acid sequence:

Human Ecto-TMEM amino acid sequence:
(SEQ ID No. 69)
THRTGLRSPDIPQDWVSFLRSFGQLTLCPRNGTVTGKWRGSHVVGLLTTL
NFGDGPDRNKTRTFQATVLGSQMGLKGSSAGQLVLITARVTTERTAGTCL
YFSAVPGILPSSQPPISCSEEGAGNATLSPRMGEECVSVWSHEGLVLTKL
LTSEELALCGSR
Murine Ecto-TMEM amino acid sequence:
(SEQ ID No. 70)
THTTGLRSPDIPQDWVSFLRSFGQLSLCPMNETVTGTWQGPHVVGLLTTL
NFGDGPDRNKTQTFQAKIHGSQIGLTGSSAGESVLVTARVASGRTPGTCL
YFSGVPKVLPSSQPPISCSEEGVGNATLSPVMGEECVRVWSHERLVLTEL
LTSEELALCGS

IGFBP3 at 50 ng/ml and ecto-TMEM219 at 130 ng/ml were added to culture medium at day+1 from mini-guts culture (see below).

Newly generated anti-IGFBP3 monoclonal antibodies were added at 1:1 molecular ratio as compared to IGFBP3 at 10 ug/ml final concentration.

Crypts Isolation and Mini-Guts Development

Humans

Crypts were extracted from mucosa and sub-mucosa of intestinal samples of healthy subjects (healthy controls) or obtained from patients with established Crohn's disease undergoing surgery for disease complications (strictures, fistulae). Mucosa was incubated with a mixture of antibiotics Normocin, [Invivogen, San Diego, Calif. 92121, USA; catalog code ant-nr], Gentamycin [Invitrogen, Carlsbad, Calif., USA catalog code ant-gn] and Fungizone [Invitrogen 15290018]) for 15 minutes at room temperature, and then tissue was minced into small pieces and incubated with 10 mM Dithiothreitol (DTT) (Sigma) in PBS 2-3 times for several minutes. Samples were then transferred to 8 mM EDTA in PBS and incubated for 30 minutes at 37° C. After this step, vigorous shaking of the sample yielded supernatants enriched in colonic crypts. Fetal bovine serum (FBS, Sigma 12103C-500ML) was added to a final concentration of 5%, and single cells were removed by centrifugation 40×g for 2 minutes. Crypts were mixed with 50 μl of Matrigel (BD Biosciences 354234) and plated on pre-warmed culture dishes. After solidification, crypts were overlaid with complete crypt culture medium: Wnt3a-conditioned medium and Advanced DMEM/F12 (Life Technologies 1263010) 50:50, supplemented with Glutamax, 10 mM (Life Technologies 35050038) HEPES (Life Technologies 15630080), N-2 [1×] (Life Technologies 17502048), B−27 without retinoic acid [1×](Life Technologies 12587010), 10 mM Nicotinamide (Sigma N0636), 1 mM N-Acetyl-L-cysteine (Sigma A965), 50 ng/ml human EGF (Life Technologies PHG0311), 1 μg/ml RSPO1 (Sino Biological 11083-H08H), 100 ng/ml human Noggin (Peprotech 12010C), 1 μg/ml Gastrin (Sigma-Aldrich SCP0152), 500 nM LY2157299 (Axon MedChem 1491), 10 μM SB202190 (Sigma S7067) and 0.01 μM PGE2 (Sigma P6532). Medium was replaced every 3 days. Purified crypts have been cultured for 8 days with/without recombinant proteins/Antibodies as described in the Recombinant proteins and interventional studies section. After 8 days, crypts were collected, and the morphology, mini-gut growth, expression of intestinal signature markers (EphB2, LGR5, h-TERT), and Caspase 8 (Life Technologies) were examined using RT-PCR.

Percentage of developed mini-guts with at least one crypt domain was assessed as already described (4,18).

Murine

Crypts were obtained from C57BL/6J mice. Briefly the colon was cut into 2-4 mm pieces with scissors and fragments were washed in 30 ml of ice-cold PBS and then incubated with 20 mM EDTA-PBS at 37° C. Finally, fragments were treated trypsin/DNAse solution to obtain crypts. After this step, vigorous shaking of the sample yielded supernatants enriched in colonic crypts. Crypts were mixed with Matrigel and plated on pre-warmed culture dishes. After solidification of matrigel (10-15 min at 37° C.), crypts were overlaid with culture medium (ADF, 10 mM HEPES, N-2, B27 without retinoic acid, 10 μM Y−27632, 1 μM JAG1 peptide (Anaspec, Fremont, Calif., USA), 1 μg/ml R-Spondin 1, 50 ng/ml EGF (Invitrogen), and 100 ng/ml Noggin (Peprotech, Rocky Hill, N.J., USA), and medium was changed every other day until day 8. After 8 days, percentage of developed mini-guts was assessed.

qRT-PCR Analysis

RNA from purified intestinal crypts was extracted using Trizol Reagent (Invitrogen), and qRT-PCR analysis was performed using TaqMan assays (Life Technologies, Grand Island, N.Y.) according to the manufacturer's instructions. The normalized expression values were determined using the ΔΔCt or the ΔCt method. Quantitative reverse transcriptase polymerase chain reaction (qRT-PCR) data were normalized for the expression of ACTB. Statistical analysis compared gene expression across all cell populations for each patient via one-way ANOVA followed by Bonferroni post-test for multiple comparisons between the population of interest and all other populations. Analysis was performed in technical and biological triplicates.

The list of genes which expression has been quantified by qRT-PCR is reported below.

| Gene | | | Band Size | Reference |
|---|---|---|---|---|
| Symbol | UniGene # | Refseq Accession # | (bp) | Position |
| LGR5 | Hs.658889 | NM_003667 | 91 | 1665 |
| EPHB2 | Hs.523329 | NM_004442 | 68 | 2908 |
| TERT | Hs.492203 | NM_198253 | 106 | 1072 |
| ACTB | Hs.520640 | NM_001101 | 174 | 730 |
| Caspase 8 | Hs.599762 | NM_001080124.1 | 124 | 648 |

Competitive ELISA Binding Assay

The following reagents were used to screen the newly generated anti-IGFBP3 antibodies: Recombinant Human IGFBP3 (0,223 mg/ml R&D System 8874-B3-025), Ecto-TMEM219 (0.5 mg/ml GenScript), newly generated anti-IGFBP3 mAbs (Trianni), anti-human IgG HRP (Life Technologies A24470), bovine serum albumin (BSA), Tween 20 (TW), ELISA colorimetricTMB reagent (HRP substrate, Item H Sigma, RABTMB3), ELISA STOP solution (Item I, Sigma, RABSTOP3). We also employed a blocking reagent solution (3% BSA in PBS) and a diluent solution (0.5% BSA, 0.05% Tw in PBS).

Microplate (Thermofisher, Electron Corporation, 2801) was coated with 50 μl/well of 4 μg/ml rhIGFBP3 dissolved in PBS or PBS alone (no coating). Plate was incubated 90 minutes at 37° C. and washed with PBS (300 μl/well) and incubated with the blocking reagent (200 μl/well) 2 hours at room temperature. Samples were then diluted in the diluent solution (50 μl/well) and added to the plate as following: diluent solution (none), ecto-TMEM219 10 μg/ml, ecto-TMEM219 10 μg/ml+ anti-IGFBP3 mAbs 10 μg/ml, anti-IGFBP3 mAbs 10 μg/ml alone. After washing steps, plate was then incubated at room temperature for 1 hour with anti 6× His tag HRP diluted 1:2000 in Diluent solution (50 μl/well). ELISA plate was then read after adding visualization solution at ELISAreader and adsorbance was measured.

Beta-Cells

Betalox-5 cells, a human beta cell line (36) were grown in culture flasks containing DMEM (glucose 1 g/L), BSA fraction V (0.02% wt/vol), Non-essential amino acids (1×) penicillin (100 units/mL), and streptomycin (100 μg/mL). The cells were cultured at 37° C. in a humidified incubator in 5% CO2. The cells were passaged once every second week. Beta cells were cultured with or without IGFBP3, with or without ecto-TMEM219, with or without newly generated monoclonal antibodies (see Recombinant proteins and interventional studies) and cells were collected for immunofluorescence studies, RNA extraction, apoptosis detection, and protein analysis. Supernatants were collected for assessment of insulin. Insulin levels were assayed with a microparticle enzyme immunoassay (Mercodia Iso-Insulin ELISA, 10-1113-01).

Statistical Analysis

Data are presented as mean and standard error of the mean (SEM) and were tested for normal distribution with the Kolmogorov-Smirnov test and for homogeneity of variances with Levene's test. The statistical significance of differences was tested with two-tailed t-test. Significance between the two groups was determined by two-tailed unpaired Student's t test. For multiple comparisons, the ANOVA test with Bonferroni correction was employed. Graphs and data were generated using GraphPad Prism version 6.0 (GraphPad Software, La Jolla, Calif.). All statistical tests were performed at the 5% significance level.

Anti-IGFBP3 mAbs Efficacy in T1D Mouse Model Following Intraperitoneal (IP) Administration

Animals

Female non-obese diabetic (NOD) mice (10 weeks old) were obtained from the Charles River Laboratories, Calco, Varese, Italy (stock #613). All mice were cared for and used in accordance with Italian law on animal care N° 116/1992 and the European Communities Council Directive EEC/609/86.

Diabetes Monitoring and Treatment

Overt diabetes (the most advanced stage, characterized by elevated fasting blood glucose concentration and classical symptoms) was defined as blood glucose levels above 250 mg/dL for three consecutive measurements. Glycemia was monitored twice a week.

Inventors set up the following treatment groups:

1) Untreated

2) Ecto-TMEM219 0.1 mg/day (i.p) for 10 days

3) Anti-IGFBP3 M1 0.5 mg/day (i.p) for 10 days

Ecto-TMEM and antibody were dissolved in PBS.

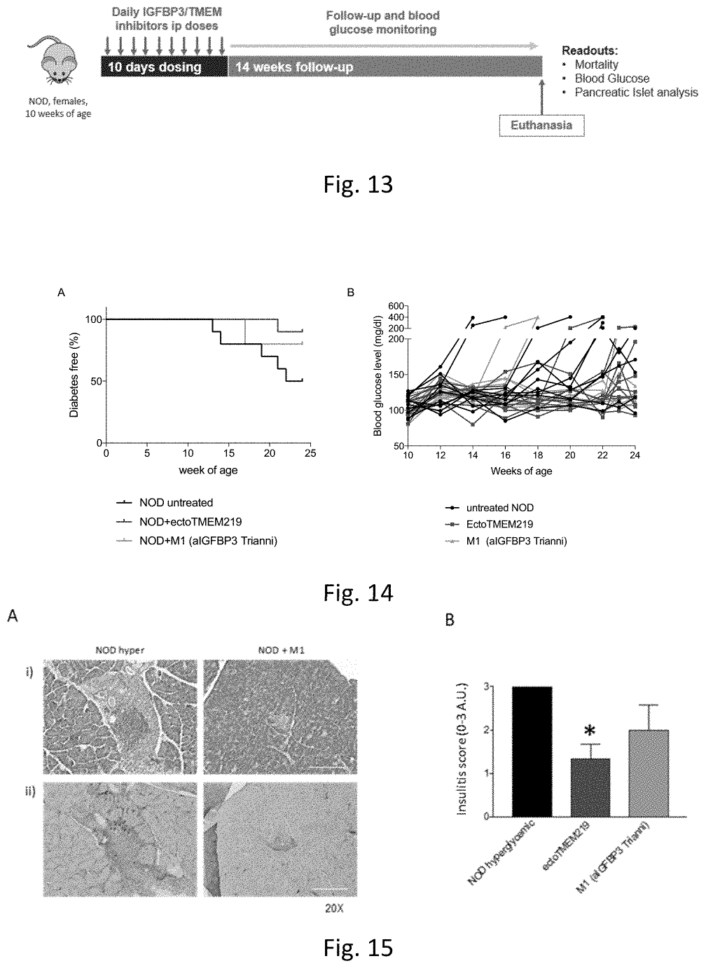

N=10 mice were included in each group of treatment. Treatment started when mice were weeks old at day 0. Mice were followed up for up to 23 weeks of age. Mice were harvested when diabetes was assessed or at week 23. Plasma samples and pancreas were collected for ex vivo analysis. The experimental timelines are described in FIG. 13.

Insulitis Scoring and Pancreatic Islet Histopathology

Insulitis scoring was performed on 5-μm-thick formalin-fixed, paraffin-embedded, hematoxylin and eosin (H&E)-stained pancreatic sections as previously described (Vergani A et al. Diabetes 2010; Ben Nasr M et al. Sci Transl Med 2017). Insulitis scoring was performed on hematoxylin and eosin (H&E) and Insulin stained pancreatic sections. A score of 0 to 4 was assigned based on islet infiltration by an experienced pathologist. Insulitis scores were graded as follows: grade 0, normal islets; grade 1, mild mononuclear infiltration (25%) at the periphery; grade 2, 25-50% of the islets infiltrated; grade 3, (50% of the islets infiltrated); grade 4, islets completely infiltrated with no residual parenchyma remaining. At least 30 islets per group were analyzed and pooled from sections obtained from different mice.

Statistical Analysis

Data are presented as mean and standard error of the mean (SEM) unless otherwise reported. Diabetes incidence among different groups was analyzed with the log-rank (Mantel-Cox) test. Statistical analysis was conducted using GraphPad Prism version 7.0 (GraphPad Software, La Jolla, Calif.). All statistical tests were performed at the 5% significance level.

### Example 1: Monoclonal Antibodies Development

Monoclonal anti-IGFBP3 antibodies were discovered through the utilization of transgenic mouse, where the relevant human immunoglobulin sequences have been introduced into the genome of the animal by genetic engineering, the Trianni Mouse™ (Trianni). Through use of such technology, chimeric monoclonal antibodies containing the full repertoire of human heavy- and light-chain variable domains and the retention of the mouse constant domains were produced.

Essentially, two cohorts of Trianni Mouse™ (Cohort 1: ALD/MDP adjuvant and Cohort 2: SAS/Ribi adjuvant) were immunized with a purified preparation of IGFPP3 antigen (lot #AB08BP1210), two injections a week for 4 weeks then 2 weeks extension. Then, lymphatic cells (such as B-cells) were recovered from the mice that express antibodies, such cells were fused with a myeloid-type cell line to prepare immortal hybridoma cell lines, and such hybridoma cell lines were screened and selected to identify hybridoma cell lines that produce antibodies specific to human IGFBP3 (lot #AB08BP1210) by ELISA. Hybridoma cell lines that were reactive for the antigen of interest were expanded. Sequencing was accomplished by RNA isolation, followed by cDNA sequencing of the human VH and human VK using Sanger sequencing methods.

Antibodies can be expressed in cell lines other than hybridoma cell lines. Sequences encoding antibodies can be used for transformation of a suitable mammalian host cell. In fact, the monoclonal antibody deriving from cohort 1, M1 was expressed in a transient gene expression system in mammalian cell.

Method of Expressing Recombinant Protein in CHO Cells

The corresponding M1 cDNAs was cloned into evitria's vector system using conventional (non-PCR based) cloning techniques to produce a fully human IgG4 mAb. The evitria vector plasmids were gene synthesized. Plasmid DNA was prepared under low-endotoxin conditions based on anion exchange chromatography. Correctness of the sequences was verified with Sanger sequencing (with up to two sequencing reactions per plasmid depending on the size of the cDNA.)

Suspension-adapted CHO K1 cells (evitria) was used for production. The seed was grown in eviGrow medium, a chemically defined, animal-component free, serum-free medium. Cells were transfected with eviFect, evitria's custom-made, proprietary transfection reagent, and cells were grown after transfection in eviMake, an animal-component free, serum-free medium, at 37° C. and 5% CO2 for 7 days. Supernatant was harvested by centrifugation and subsequent filtration (0.2 μm filter).

The antibody was purified using MabSelect™ SuRe™ with Dulbecco's PBS (Lonza BE17-512Q) as wash buffer and 0.1 M Glycine pH 3.5 as elution buffer. Subsequent size exclusion chromatography was performed on a HiLoad Superdex 200 pg column using the final buffer as running buffer.

Monomericity was determined by analytical size exclusion chromatography with an Agilent AdvanceBio SEC column (300A 2.7 um 7.8×300 mm) and DPBS as running buffer at 0.8 ml/min. Remarkably, the monomericity of M1 was >95%, exhibiting only <5% of aggregated. The high monomericity of the protein is an exceptional property that should aid in its manufacture.

Affinity Measurement

Octet BLI-Based Analysis

Antibodies possessed high affinity to the target. The binding affinity measurements were performed using an Octet instrument (Octet BMIA), which is a Biolayer Interferometry (BLI) platform based on Biomolecular Interaction Analysis. To establish the assay, the target monoclonal antibody (30 μg/ml in PBS) was immobilized via Fc on the via Anti-Mouse IgG Fc Capture (AMC) or Anti-Human IgG Fc Capture (AMC) biosensors and the interaction with the antigen, human IGFBP3 (R&D, cat n° 675 B3) at 150 nM was measured.

The affinity measurement of the anti-IGFBP3 mAbs for the target human IGFBP3 are reported in Table 1.

TABLE 1
Affinity measurement of exemplified antibodies

| Antibody | KD (M) |
|---|---|
| E01 | 1.2E−09 |
| E02 | >1.0E−12 |
| E08 | 6.1E−10 |
| E14 | 8.5E−10 |
| E19 | 6.9E−10 |
| E20 | 8.5E−10 |
| E23 | 1.1E−09 |
| E24 | >1.0E−12 |
| M1 | >1.0E−12 |

The sequences of the 9 novel anti-IGFBP3 antibodies are reported in Tables 2-5 below.

TABLE 2
VH CDR Sequences of exemplified antibodies

| Antibody | CDR1 | CDR2 | CDR3 |
|---|---|---|---|
| E01 | GFTFSSYG | ISYDGSNK | ARGGEYFYYYGLDV |
| | (SEQ ID No. 1) | (SEQ ID No. 2) | (SEQ ID No. 3) |
| E02 | GYTFSNYG | INTYNGNT | ARDRGYSSSPYYYYYGMDV |
| | (SEQ ID No. 4) | (SEQ ID No. 5) | (SEQ ID No. 6) |
| E08 | GYTFSNYG | INTYNGNT | ARDRGYSSSPYYYYYGMDV |
| | (SEQ ID No. 4) | (SEQ ID No. 5) | (SEQ ID No. 6) |
| E14 | GYTFSNYG | INTYNGNT | ARDRGYSSSPYYYYYGMDV |
| | (SEQ ID No. 4) | (SEQ ID No. 5) | (SEQ ID No. 6) |
| E19 | GFTFSSYG | ISYDGSNK | ARGGEYFYYYGLDV |
| | (SEQ ID No. 1) | (SEQ ID No. 2) | (SEQ ID No. 3) |
| E20 | GYTFSNYG | INTYNGNT | ARDRGYSSSPYYYYYGMDV |
| | (SEQ ID No. 4) | (SEQ ID No. 5) | (SEQ ID No. 6) |
| E23 | GFTFSSYG | ISYDGSNK | ARGGEYFYYYGLDV |
| | (SEQ ID No. 1) | (SEQ ID No. 2) | (SEQ ID No. 3) |
| E24 | GYTFTNYG | INAYNGNT | ARDRGYSSSPYYYYYGMDV |
| | (SEQ ID No. 7) | (SEQ ID No. 8) | (SEQ ID No. 6) |
| M1 | GGSISTYY (SEQ | IYYSGST (SEQ | ARYDIVTGYPHYYYYVMDV |
| | ID No. 9) | ID No. 10) | (SEQ ID No. 11) |

TABLE 3
VL CDR sequences of exemplified antibodies

| Antibody | CDR1 | CDR2 | CDR3 |
|---|---|---|---|
| E01 | QSVSSSS (SEQ ID | GAS (SEQ | QQDYNLPLT (SEQ ID No. |
| | No. 12) | ID No. 13) | 14) |
| E02 | QSVSSSH (SEQ ID | GAS (SEQ | QQDYNLTIT (SEQ ID No. |
| | No. 15) | ID No. 13) | 16) |
| E08 | QSVSSSS (SEQ ID | GAS (SEQ | QQDYNLPLT (SEQ ID No. |
| | No. 12) | ID No. 13) | 14) |
| E14 | QGISNY (SEQ ID | AAS (SEQ | QQYNSYPFT (SEQ ID No. |
| | No. 17) | ID No. 18) | 19) |
| E19 | QGISSA (SEQ ID | DAS (SEQ | QQFNNYPST (SEQ ID |
| | No. 20) | ID No. 21) | No. 22) |
| E20 | QGIRND (SEQ ID | AAS (SEQ | LQHNSYPYT (SEQ ID No. |
| | No. 23) | ID No. 18) | 24) |
| E23 | QGISNY (SEQ ID | AAS (SEQ | QQYNSYPFT (SEQ ID No. |
| | No. 17) | ID No. 18) | 19) |
| E24 | QGIRNA (SEQ ID | AAS (SEQ | LQDYNYPLT (SEQ ID No. |
| | No. 25) | ID No. 18) | 26) |
| M1 | RGIRNA (SEQ ID | AAS (SEQ | LQDYNYPLT (SEQ ID No. |
| | No. 27) | ID No. 18) | 26) |

CDR definition is provided using annotation tool from http://www.abysis.org/ based on full VH and VL amino acid sequences as defined in Tables 4 and 5.

For example, the VH amino acid sequence of any antibody disclosed herein is plugged into the annotation tool and Kabat defined CDR sequences, or IMGT, or Chothia, or AbM or Contact defined CDR sequences are provided. Using the “All, side by side” feature, defined CDR sequences are provided. The following example is based on SEQ ID No. 36 and 45.

TABLE 3.1
All, side by side defined CDR sequences of VH (SEQ ID No. 36) and VL (SEQ ID No. 45):
Regions Definition - All, side by side

| Region | Definition | Sequence Fragment | Residues |
|---|---|---|---|
| HFR1 | Chothia | QVQLQESGPGLVKPSETLSLTCTVS----- (SEQ ID No. 71) | 1-25 |
| | AbM | QVQLQESGPGLVKPSETLSLTCTVS-----(SEQ ID No. 71) | 1-25 |
| | Kabat | QVQLQESGPGLVKPSETLSLTCTVSGGSIS(SEQ ID No. 72) | 1-30 |
| | Contact | QVQLQESGPGLVKPSETLSLTCTVSGGSI-(SEQ ID No. 73) | 1-29 |
| | IMGT | QVQLQESGPGLVKPSETLSLTCTVS-----(SEQ ID No. 71) | 1-25 |
| CDR-H1 | Chothia | GGSISTY---(SEQ ID No. 74) | 26-32 |
| | AbM | GGSISTYYWS (SEQ ID No. 75) | 26-35 |
| | Kabat | -----TYYWS (SEQ ID No. 76) | 31-35 |
| | Contact | ----STYYWS (SEQ ID No. 77) | 30-35 |
| | IMGT | GGSISTYY-(SEQ ID No. 9) | 26-33 |
| HFR2 | Chothia | YWSWIRQPPGKGLEWIGYI (SEQ ID No. 78) | 33-51 |
| | AbM | ---WIRQPPGKGLEWIG-- (SEQ ID No. 79) | 36-49 |
| | Kabat | ---WIRQPPGKGLEWIG-- (SEQ ID No. 79) | 36-49 |
| | Contact | ---WIRQPPGKGLE-----(SEQ ID No. 80) | 36-46 |
| | IMGT | -WSWIRQPPGKGLEWIGY- (SEQ ID No. 81) | 34-50 |
| CDR-H2 | Chothia | -----YYSGS--------- (SEQ ID No. 82) | 52-56 |
| | AbM | ---YIYYSGSTN------ (SEQ ID No. 83) | 50-58 |
| | Kabat | ---YIYYSGSTNYNPSLKS (SEQ ID No. 84) | 50-65 |
| | Contact | WIGYIYYSGSTN-------(SEQ ID No. 85) | 47-58 |
| | IMGT | ----IYYSGST--------(SEQ ID No. 10) | 51-57 |
| HFR3 | Chothia | TNYNPSLKSRVTISVDTSKNQFSLKLSSVTAADTAVYYCAR (SEQ | 57-97 |
| | | ID No. 86) | |
| | AbM | --YNPSLKSRVTISVDTSKNQFSLKLSSVTAADTAVYYCAR | 59-97 |
| | | (SEQ ID No. 87) | |
| | Kabat | ---------RVTISVDTSKNQFSLKLSSVTAADTAVYYCAR (SEQ | 66-97 |
| | | ID No. 88) | |
| | Contact | --YNPSLKSRVTISVDTSKNQFSLKLSSVTAADTAVYYC- | 59-95 |
| | | (SEQ ID No. 89) | |
| | IMGT | -NYNPSLKSRVTISVDTSKNQFSLKLSSVTAADTAVYYC- | 58-95 |
| | | (SEQ ID No. 90) | |
| CDR-H3 | Chothia | --YDIVTGYPHYYYYVMDV (SEQ ID No. 91) | 98-114 |
| | AbM | --YDIVTGYPHYYYYVMDV (SEQ ID No. 91) | 98-114 |
| | Kabat | --YDIVTGYPHYYYYVMDV (SEQ ID No. 91) | 98-114 |
| | Contact | ARYDIVTGYPHYYYYVMD- (SEQ ID No. 92) | 96-113 |
| | IMGT | ARYDIVTGYPHYYYYVMDV (SEQ ID No. 11) | 96-114 |
| HFR4 | Chothia | -WGQGTTVTVSS (SEQ ID No. 93) | 115-125 |
| | AbM | -WGQGTTVTVSS (SEQ ID No. 93) | 115-125 |
| | Kabat | -WGQGTTVTVSS (SEQ ID No. 93) | 115-125 |
| | Contact | VWGQGTTVTVSS (SEQ ID No. 94) | 114-125 |
| | IMGT | -WGQGTTVTVSS (SEQ ID No. 93) | 115-125 |
| LFR1 | Chothia | AIQMTQSPSSLSASVGDRVTITC------(SEQ ID No. 95) | 1-23 |
| | AbM | AlOMTOSPSSLSASVGDRVTITC------(SEQ ID No. 95) | 1-23 |
| | Kabat | AIQMTQSPSSLSASVGDRVTITC------(SEQ ID No. 95) | 1-23 |
| | Contact | AIQMTQSPSSLSASVGDRVTITCRASRGI (SEQ ID No. 96) | 1-29 |
| | IMGT | AIQMTQSPSSLSASVGDRVTITCRAS---(SEQ ID No. 97) | 1-26 |
| CDR-L1 | Chothia | RASRGIRNALG--(SEQ ID No. 98) | 24-34 |
| | AbM | RASRGIRNALG--(SEQ ID No. 98) | 24-34 |
| | Kabat | RASRGIRNALG--(SEQ ID No. 98) | 24-34 |
| | Contact | ------RNALGWY (SEQ ID No. 99) | 30-36 |
| | IMGT | ---RGIRNA----(SEQ ID No. 27) | 27-32 |
| LFR2 | Chothia | --WYQQKPGTAPKLLIY (SEQ ID No. 100) | 35-49 |
| | AbM | --WYQQKPGTAPKLLIY (SEQ ID No. 100) | 35-49 |
| | Kabat | --WYQQKPGTAPKLLIY (SEQ ID No. 100) | 35-49 |
| | Contact | ----QQKPGTAPK----(SEQ ID No. 101) | 37-45 |
| | IMGT | LGWYQQKPGTAPKLLIY (SEQ ID No. 102) | 33-49 |
| CDR-L2 | Chothia | ----AASSLQS (SEQ ID No. 103) | 50-56 |
| | AbM | ----AASSLQS (SEQ ID No. 103) | 50-56 |
| | Kabat | ----AASSLQS (SEQ ID No. 103) | 50-56 |
| | Contact | LLIYAASSLQ- (SEQ ID No. 104) | 46-55 |
| | IMGT | ----AA----- | 50-51 |
| LFR3 | Chothia | -----GVPSRFSGSGSGTDFTLTISSLQPEDSATYYC (SEQ ID | 57-88 |
| | | No. 105) | |
| | AbM | -----GVPSRFSGSGSGTDFTLTISSLQPEDSATYYC (SEQ ID | 57-88 |
| | | No. 105) | |
| | Kabat | -----GVPSRFSGSGSGTDFTLTISSLQPEDSATYYC (SEQ ID | 57-88 |
| | | No. 105) | |
| | Contact | ----SGVPSRFSGSGSGTDFTLTISSLQPEDSATYYC (SEQ ID | 56-88 |
| | | No. 106) | |
| | IMGT | SSLQSGVPSRFSGSGSGTDFTLTISSLQPEDSATYYC (SEQ ID | 52-88 |
| | | No. 107) | |
| CDR-L3 | Chothia | LQDYNYPLT (SEQ ID No. 26) | 89-97 |
| | AbM | LQDYNYPLT (SEQ ID No. 26) | 89-97 |
| | Kabat | LQDYNYPLT (SEQ ID No. 26) | 89-97 |
| | Contact | LQDYNYPL-(SEQ ID No. 108) | 89-96 |
| | IMGT | LQDYNYPLT (SEQ ID No. 26) | 89-97 |
| LFR4 | Chothia | -FGGGTKVEIK (SEQ ID No. 109) | 98-107 |
| | AbM | -FGGGTKVEIK (SEQ ID No. 109) | 98-107 |
| | Kabat | -FGGGTKVEIK (SEQ ID No. 109) | 98-107 |
| | Contact | TFGGGTKVEIK (SEQ ID No. 110) | 97-107 |
| | IMGT | -FGGGTKVEIK (SEQ ID No. 109) | 98-107 |

TABLE 4
VH amino acid sequences of exemplified antibodies

| Antibody | AA of VH |
|---|---|
| E01 | QVQLVESGGGWQPGRSLRLSCAASGFTFSSYGMHWVRQAPGKGLE |
| | WVAVISYDGSNKNYVDSVKGRFTISRDNSKNTLYLQMNSLRAEDTAVY |
| | YCARGGEYFYYYGLDVWGQGTTVTVSS (SEQ ID No. 28) |
| E02 | QVQLVQSGAEVKKPGASVKVSCKASGYTFSNYGISWVRQAPGQGLE |
| | WMGWINTYNGNTNYAQKLQGRVTMTTDTSTSTAYMALRGLRSDDTA |
| | VYYCARDRGYSSSPYYYYYGMDVWGQGTTVTVSS (SEQ ID No. 29) |
| E08 | QVQLVQSGAEVKKPGASVKVSCKASGYTFSNYGISWVRQAPGQGLE |
| | WMGWINTYNGNTNYAQKLQGRVTMTTDTSTSTAYMALRGLRSDDTA |
| | VYYCARDRGYSSSPYYYYYGMDVWGQGTTVTVSS (SEQ ID No. 30) |
| E14 | QVQLVQSGAEVKKPGASVKVSCKASGYTFSNYGISWVRQAPGQGLE |
| | WMGWINTYNGNTNYAQKLQGRVTMTTDTSTSTAYMALRGLRSDDTA |
| | VYYCARDRGYSSSPYYYYYGMDVWGQGTTVTVSS (SEQ ID No. 31) |
| E19 | QVQLVESGGGWQPGRSLRLSCAASGFTFSSYGMHWVRQAPGKGLE |
| | WVAVISYDGSNKNYVDSVKGRFTISRDNSKNTLYLQMNSLRAEDTAVY |
| | YCARGGEYFYYYGLDVWGQGTTVTVSS (SEQ ID No. 32) |
| E20 | QVQLVQSGAEVKKPGASVKVSCKASGYTFSNYGISWVRQAPGQGLE |
| | WMGWINTYNGNTNYAQKLQGRVTMTTDTSTSTAYMALRGLRSDDTA |
| | VYYCARDRGYSSSPYYYYYGMDVWGQGTTVTVSS (SEQ ID No. 33) |
| E23 | QVQLVESGGGWQPGRSLRLSCAASGFTFSSYGMHWVRQAPGKGLE |
| | WVAVISYDGSNKNYWDSVKGRFTISRDNSKNTLYLQMNSLRAEDTAV |
| | YYCARGGEYFYYYGLDVWGQGTTVTVSS (SEQ ID No. 34) |
| E24 | QVQLVQSGAEVKKPGASVKVSCKASGYTFTNYGISWRQAPGQGLE |
| | WMGWINAYNGNTNYAQKLQGRVTMTTVTYTSTAYMELRSLRSDDTA |
| | VYYCARDRGYSSSPYYYYYGMDVWGQGTTVTVSS (SEQ ID No. 35) |
| M1 | QVQLQESGPGLVKPSETLSLTCTVSGGSISTYYWSWIRQPPGKGLEWI |
| | GYIYYSGSTNYNPSLKSRVTISVDTSKNQFSLKLSSVTAADTAVYYCAR |
| | YDIVTGYPHYYYYVMDVWGQGTTVTVSS (SEQ ID No. 36) |

TABLE 5
VL amino acid sequences of exemplified antibodies

| Antibody | AA OF VK |
|---|---|
| E01 | EIVMTQSPATLSLSPGERATLSCRASQSVSSSSLSWYQQKPGQAPR |
| | LLIYGASTRATGIPARFSGSGSGTDFTLTISSLQPEDFAVYYCQQDYN |
| | LPLTFGGGTKVEIK (SEQ ID No. 37) |
| E02 | EIVMTQSPATLSLSPGERATLSCRASQSVSSSHLSWYQQKPGQAPR |
| | LLIYGASTRATGIPARFSGSGSGTDFTLTISSLQPEDFAVYYCQQDYN |
| | LTITFGQGTRLEIK (SEQ ID No. 38) |
| E08 | EIVMTQSPATLSLSPGERATLSCRASQSVSSSSLSWYQQKPGQAPR |
| | LLIYGASTRATGIPARFSGSGSGTDFTLTISSLQPEDFAVYYCQQDYN |
| | LPLTFGGGTKVEIK (SEQ ID No. 39) |
| E14 | DIQMTQSPSSLSASIGDRVTITCRASQGISNYLAWFQQKPGKAPKSLI |
| | YAASSLQSGVPSKFSGSGSGTDFTLTISSLQPEDFATYYCQQYNSYP |
| | FTFGPGTKVDIK (SEQ ID No. 40) |
| E19 | AIQLTQSPSSLSASVGDRVTITCRAGQGISSALAWYQQKPGKAPKILIY |
| | DASSLESGVPSRFSGSGSGTDFTLTISSLQPEDFATYYCQQFNNYPS |
| | TFGQGTKLEIK (SEQ ID No. 41) |
| E20 | DIQMTQSPSSLSASVGDRVTITCRASQGIRNDLGWYQQKPGKAPKRL |
| | IYAASSLQSGVPSRFSGSGSGTEFTLTISSLQPEDFATYYCLQHNSYP |
| | YTFGQGTKLEIK (SEQ ID No. 42) |
| E23 | DIQMTQSPSSLSASIGDRVTITCRASQGISNYLAWFQQKPGKAPKSLI |
| | YAASSLQSGVPSKFSGSGSGTDFTLTISSLQPEDFATYYCQQYNSYP |
| | FTFGPGTKVDIK (SEQ ID No. 43) |
| E24 | AIQMTQSPSSLSASVGDKVTITCRASQGIRNALGWYQQKPGTAPKLLI |
| | YAASSLQSGVPSRFSGSGSGTDFTLTISSLQPEDSATYYCLQDYNYP |
| | LTFGGGTKVEIK (SEQ ID No. 44) |
| M1 | AIQMTQSPSSLSASVGDRVTITCRASRGIRNALGWYQQKPGTAPKLLI |
| | YAASSLQSGVPSRFSGSGSGTDFTLTISSLQPEDSATYYCLQDYNYP |
| | LTFGGGTKVEIK (SEQ ID No. 45) |

TABLE 6
VH nucleotide sequences of exemplified antibodies

| Antibody | DNA of VH |
|---|---|
| E01 | CAGGTGCAGCTGGTGGAGTCTGGGGGAGGCGTGGTCCAGCCTGG |
| | GAGGTCCCTGAGACTCTCCTGTGCAGCCTCTGGATTCACCTTCAGT |
| | AGCTATGGCATGCACTGGGTCCGCCAGGCTCCAGGCAAGGGGCT |
| | GGAGTGGGTGGCAGTTATATCATATGATGGAAGTAATAAAAACTAT |
| | GTAGACTCCGTGAAGGGCCGATTCACCATCTCCAGAGACAATTCCA |
| | AGAACACGCTGTATCTGCAAATGAACAGCCTGAGAGCTGAGGACA |
| | CGGCTGTGTATTACTGTGCGAGAGGAGGGGAGTACTTCTACTATTA |
| | CGGTTTGGACGTCTGGGGCCAAGGGACCACGGTCACCGTCTCCTC |
| | A (SEQ ID No. 46) |
| E02 | CAGGTTCAGCTGGTGCAGTCTGGAGCTGAGGTGAAGAAGCCTGGG |
| | GCCTCAGTGAAGGTCTCCTGCAAGGCTTCTGGTTACACCTTTTCCA |
| | ATTATGGTATCAGCTGGGTGCGACAGGCCCCTGGACAAGGGCTTG |
| | AGTGGATGGGATGGATCAACACTTACAATGGTAACACAAACTATGC |
| | ACAGAAGCTCCAGGGCAGAGTCACCATGACCACTGACACATCCAC |
| | GAGCACAGCCTACATGGCGCTGAGGGGCCTGAGATCTGACGACAC |
| | GGCCGTGTATTATTGTGCGAGAGATAGGGGGTATAGCAGCAGCCC |
| | TTACTACTACTACTACGGAATGGACGTCTGGGGCCAAGGGACCAC |
| | GGTCACCGTCTCCTCA (SEQ ID No. 47) |
| E08 | CAGGTTCAGCTGGTGCAGTCTGGAGCTGAGGTGAAGAAGCCTGGG |
| | GCCTCAGTGAAGGTCTCCTGCAAGGCTTCTGGTTACACCTTTTCCA |
| | ATTATGGTATCAGCTGGGTGCGACAGGCCCCTGGACAAGGGCTTG |
| | AGTGGATGGGATGGATCAACACTTACAATGGTAACACAAACTATGC |
| | ACAGAAGCTCCAGGGCAGAGTCACCATGACCACTGACACATCCAC |
| | GAGCACAGCCTACATGGCGCTGAGGGGCCTGAGATCTGACGACAC |
| | GGCCGTGTATTATTGTGCGAGAGATAGGGGGTATAGCAGCAGCCC |
| | TTACTACTACTACTACGGAATGGACGTCTGGGGCCAAGGGACCAC |
| | GGTCACCGTCTCCTCA (SEQ ID No. 48) |
| E14 | CAGGTTCAGCTGGTGCAGTCTGGAGCTGAGGTGAAGAAGCCTGGG |
| | GCCTCAGTGAAGGTCTCCTGCAAGGCTTCTGGTTACACCTTTTCCA |
| | ATTATGGTATCAGCTGGGTGCGACAGGCCCCTGGACAAGGGCTTG |
| | AGTGGATGGGATGGATCAACACTTACAATGGTAACACAAACTATGC |
| | ACAGAAGCTCCAGGGCAGAGTCACCATGACCACTGACACATCCAC |
| | GAGCACAGCCTACATGGCGCTGAGGGGCCTGAGATCTGACGACAC |
| | GGCCGTGTATTATTGTGCGAGAGATAGGGGGTATAGCAGCAGCCC |
| | TTACTACTACTACTACGGAATGGACGTCTGGGGCCAAGGGACCAC |
| | GGTCACCGTCTCCTCA (SEQ ID No. 49) |
| E19 | CAGGTGCAGCTGGTGGAGTCTGGGGGAGGCGTGGTCCAGCCTGG |
| | GAGGTCCCTGAGACTCTCCTGTGCAGCCTCTGGATTCACCTTCAGT |
| | AGCTATGGCATGCACTGGGTCCGCCAGGCTCCAGGCAAGGGGCT |
| | GGAGTGGGTGGCAGTTATATCATATGATGGAAGTAATAAAAACTAT |
| | GTAGACTCCGTGAAGGGCCGATTCACCATCTCCAGAGACAATTCCA |
| | AGAACACGCTGTATCTGCAAATGAACAGCCTGAGAGCTGAGGACA |
| | CGGCTGTGTATTACTGTGCGAGAGGAGGGGAGTACTTCTACTATTA |
| | CGGTTTGGACGTCTGGGGCCAAGGGACCACGGTCACCGTCTCCTC |
| | A (SEQ ID No. 50) |
| E20 | CAGGTTCAGCTGGTGCAGTCTGGAGCTGAGGTGAAGAAGCCTGGG |
| | GCCTCAGTGAAGGTCTCCTGCAAGGCTTCTGGTTACACCTTTTCCA |
| | ATTATGGTATCAGCTGGGTGCGACAGGCCCCTGGACAAGGGCTTG |
| | AGTGGATGGGATGGATCAACACTTACAATGGTAACACAAACTATGC |
| | ACAGAAGCTCCAGGGCAGAGTCACCATGACCACTGACACATCCAC |
| | GAGCACAGCCTACATGGCGCTGAGGGGCCTGAGATCTGACGACAC |
| | GGCCGTGTATTATTGTGCGAGAGATAGGGGGTATAGCAGCAGCCC |
| | TTACTACTACTACTACGGAATGGACGTCTGGGGCCAAGGGACCAC |
| | GGTCACCGTCTCCTCA (SEQ ID No. 51) |
| E23 | CAGGTGCAGCTGGTGGAGTCTGGGGGAGGCGTGGTCCAGCCTGG |
| | GAGGTCCCTGAGACTCTCCTGTGCAGCCTCTGGATTCACCTTCAGT |
| | AGCTATGGCATGCACTGGGTCCGCCAGGCTCCAGGCAAGGGGCT |
| | GGAGTGGGTGGCAGTTATATCATATGATGGAAGTAATAAAAACTAT |
| | GTAGACTCCGTGAAGGGCCGATTCACCATCTCCAGAGACAATTCCA |
| | AGAACACGCTGTATCTGCAAATGAACAGCCTGAGAGCTGAGGACA |
| | CGGCTGTGTATTACTGTGCGAGAGGAGGGGAGTACTTCTACTATTA |
| | CGGTTTGGACGTCTGGGGCCAAGGGACCACGGTCACCGTCTCCTC |
| | A (SEQ ID No. 52) |
| E24 | CAGGTTCAGCTGGTGCAGTCTGGAGCTGAGGTGAAGAAGCCTGGA |
| | GCCTCAGTGAAGGTCTCCTGCAAGGCTTCTGGTTACACCTTTACCA |
| | ACTATGGTATCAGCTGGGTGCGACAGGCCCCTGGACAAGGGCTTG |
| | AGTGGATGGGATGGATCAACGCTTACAATGGTAACACAAACTATGC |
| | ACAGAAGCTCCAGGGCAGAGTCACCATGACCACAGTCACATACAC |
| | GAGTACAGCCTACATGGAGCTGAGGAGCCTGAGATCTGACGACAC |
| | GGCCGTGTATTACTGTGCGAGAGATAGGGGGTATAGCAGCAGCCC |
| | TTATTACTACTACTACGGTATGGACGTCTGGGGCCAAGGGACCACG |
| | GTCACCGTCTCCTCA (SEQ ID No. 53) |
| M1 | CAGGTGCAGCTGCAGGAGTCGGGCCCAGGACTGGTGAAGCCTTC |
| | GGAGACCCTGTCCCTCACCTGCACTGTCTCTGGTGGCTCCATCAGT |
| | ACTTACTACTGGAGCTGGATCCGGCAGCCCCCAGGGAAGGGACTG |
| | GAGTGGATTGGGTATATCTATTACAGTGGGAGCACCAACTACAACC |
| | CCTCCCTCAAGAGTCGAGTCACCATATCAGTAGACACGTCCAAGAA |
| | CCAGTTCTCCCTGAAGCTGAGCTCTGTGACCGCTGCGGACACGGC |
| | CGTTTATTACTGTGCGAGGTACGATATTGTGACTGGTTATCCTCACT |
| | ACTACTACTACGTTATGGACGTCTGGGGCCAAGGGACCACGGTCA |
| | CCGTCTCCTCA (SEQ ID No. 54) |

TABLE 7
VL nucleotide sequences of exemplified antibodies

| Antibody | DNA ofVL |
|---|---|
| E01 | GAAATTGTAATGACACAGTCTCCAGCCACCCTGTCTTTGTCTCCAG |
| | GGGAAAGAGCCACCCTCTCCTGCAGGGCCAGTCAGAGTGTTAGCA |
| | GCAGCTCCTTATCCTGGTACCAGCAGAAACCTGGGCAGGCTCCCA |
| | GGCTCCTCATCTATGGTGCATCCACCAGGGCCACTGGCATCCCAG |
| | CCAGGTTCAGTGGCAGTGGGTCTGGGACAGACTTCACTCTCACCAT |
| | CAGCAGCCTGCAGCCTGAAGATTTTGCAGTTTATTACTGTCAGCAG |
| | GATTATAACTTACCGCTCACTTTCGGCGGAGGGACCAAGGTGGAGA |
| | TCAAA (SEQ ID No. 55) |
| E02 | GAAATTGTAATGACACAGTCTCCAGCCACCCTGTCTTTGTCTCCAG |
| | GGGAAAGAGCCACCCTCTCCTGCAGGGCCAGTCAGAGTGTTAGCA |
| | GCAGCCATTTATCCTGGTACCAGCAGAAACCTGGGCAGGCTCCCA |
| | GGCTCCTCATCTATGGTGCATCCACCAGGGCCACTGGCATCCCAG |
| | CCAGGTTCAGTGGCAGTGGGTCTGGGACAGACTTCACTCTCACCAT |
| | CAGCAGCCTGCAGCCTGAAGATTTTGCAGTTTATTATTGTCAGCAG |
| | GATTATAATTTAACGATCACCTTCGGCCAAGGGACACGACTGGAGA |
| | TTAAA (SEQ ID No. 56) |
| E08 | GAAATTGTAATGACACAGTCTCCAGCCACCCTGTCTTTGTCTCCAG |
| | GGGAAAGAGCCACCCTCTCCTGCAGGGCCAGTCAGAGTGTTAGCA |
| | GCAGCTCCTTATCCTGGTACCAGCAGAAACCTGGGCAGGCTCCCA |
| | GGCTCCTCATCTATGGTGCATCCACCAGGGCCACTGGCATCCCAG |
| | CCAGGTTCAGTGGCAGTGGGTCTGGGACAGACTTCACTCTCACCAT |
| | CAGCAGCCTGCAGCCTGAAGATTTTGCAGTTTATTACTGTCAGCAG |
| | GATTATAACTTACCGCTCACTTTCGGCGGAGGGACCAAGGTGGAGA |
| | TCAAA (SEQ ID No. 57) |
| E14 | GACATCCAGATGACCCAGTCTCCATCCTCACTGTCTGCATCTATAG |
| | GAGACAGAGTCACCATCACTTGTCGGGCGAGTCAGGGCATTAGCA |
| | ATTATTTAGCCTGGTTTCAGCAGAAACCAGGGAAAGCCCCTAAGTC |
| | CCTGATCTATGCTGCATCCAGTTTGCAAAGTGGGGTCCCATCAAAG |
| | TTCAGCGGCAGTGGATCTGGGACAGATTTCACTCTCACCATCAGCA |
| | GCCTGCAGCCTGAAGATTTTGCAACTTATTACTGCCAACAGTATAAT |
| | AGTTACCCATTCACTTTCGGCCCTGGGACCAAAGTGGATATCAAA |
| | (SEQ ID No. 58) |
| E19 | GCCATCCAGTTGACCCAGTCTCCATCCTCCCTGTCTGCATCTGTAG |
| | GAGACAGAGTCACCATCACTTGCCGGGCAGGTCAGGGCATTAGCA |
| | GTGCTTTAGCCTGGTATCAGCAGAAACCAGGGAAAGCTCCTAAGAT |
| | CCTGATCTATGATGCCTCCAGTTTGGAAAGTGGGGTCCCATCAAGG |
| | TTCAGCGGCAGTGGATCGGGGACAGATTTCACTCTCACCATCAGCA |
| | GCCTGCAGCCTGAAGATTTTGCAACTTATTACTGTCAACAGTTTAAT |
| | AATTACCCTAGCACTTTTGGCCAGGGGACCAAGCTGGAGATCAAA |
| | (SEQ ID No. 59) |
| E20 | GACATCCAGATGACCCAGTCTCCATCCTCCCTGTCTGCATCTGTAG |
| | GAGACAGAGTCACCATCACTTGCCGGGCAAGTCAGGGCATTAGAA |
| | ATGATTTAGGCTGGTATCAGCAGAAACCAGGGAAAGCCCCTAAGCG |
| | CCTGATCTATGCTGCATCCAGTTTGCAAAGTGGGGTCCCATCAAGG |
| | TTCAGCGGCAGTGGATCTGGGACAGAATTCACTCTCACAATCAGCA |
| | GCCTGCAGCCTGAAGATTTTGCAACTTATTACTGTCTACAGCATAAT |
| | AGTTACCCGTACACTTTTGGCCAGGGGACCAAGCTGGAGATCAAA |
| | (SEQ ID No. 60) |
| E23 | GACATCCAGATGACCCAGTCTCCATCCTCACTGTCTGCATCTATAG |
| | GAGACAGAGTCACCATCACTTGTCGGGCGAGTCAGGGCATTAGCA |
| | ATTATTTAGCCTGGTTTCAGCAGAAACCAGGGAAAGCCCCTAAGTC |
| | CCTGATCTATGCTGCATCCAGTTTGCAAAGTGGGGTCCCATCAAAG |
| | TTCAGCGGCAGTGGATCTGGGACAGATTTCACTCTCACCATCAGCA |
| | GCCTGCAGCCTGAAGATTTTGCAACTTATTACTGCCAACAGTATAAT |
| | AGTTACCCATTCACTTTCGGCCCTGGGACCAAAGTGGATATCAAA |
| | (SEQ ID No. 61) |
| E24 | GCCATCCAGATGACCCAGTCTCCATCCTCCCTGTCTGCATCTGTAG |
| | GAGACAAAGTCACCATCACTTGCCGGGCAAGTCAGGGCATTAGAAA |
| | TGCTTTAGGCTGGTATCAGCAGAAACCAGGAACAGCCCCTAAACTC |
| | CTGATCTATGCTGCATCCAGTTTACAGAGTGGGGTCCCATCAAGGT |
| | TCAGCGGCAGTGGATCTGGCACAGATTTCACTCTCACCATCAGCAG |
| | CCTGCAGCCTGAAGATTCTGCAACTTATTACTGTCTACAAGATTACA |
| | ATTACCCGCTCACTTTCGGCGGAGGGACCAAGGTGGAGATCAAA |
| | (SEQ ID No. 62) |
| M1 | GCCATCCAGATGACCCAGTCTCCATCCTCCCTGTCTGCATCTGTAG |
| | GAGACAGAGTCACCATCACTTGCCGGGCAAGTCGGGGCATTAGAA |
| | ATGCTTTAGGCTGGTATCAGCAGAAACCAGGAACAGCCCCTAAACT |
| | CCTGATCTATGCTGCATCCAGTTTACAGAGTGGGGTCCCATCAAGG |
| | TTCAGCGGCAGTGGATCTGGCACAGATTTCACTCTCACCATCAGCA |
| | GCCTGCAGCCTGAAGATTCTGCAACTTATTACTGTCTACAAGATTAC |
| | AATTACCCGCTCACTTTCGGCGGAGGGACCAAGGTGGAGATCAAA |
| | (SEQ ID No. 63) |

TABLE 8
Constant region amino acid sequences

| Constant region | AA |
|---|---|
| Human IgG4 heavy | ASTKGPSVFPLAPCSRSTSESTAALGCLVKDYFPEPVTVSWNSGA |
| chain | LTSGVHTFPAVLQSSGLYSLSSWTVPSSSLGTKTYTCNVDHKPS |
| P01861.1 | NTKVDKRVESKYGPPCPSCPAPEFLGGPSVFLFPPKPKDTLMISR |
| | TPEVTCVWDVSQEDPEVQFNWYVDGVEVHNAKTKPREEQFNST |
| | YRWSVLTVLHQDWLNGKEYKCKVSNKGLPSSIEKTISKAKGQPR |
| | EPQVYTLPPSQEEMTKNQVSLTCLVKGFYPSDIAVEWESNGQPEN |
| | NYKTTPPVLDSDGSFFLYSRLTVDKSRWQEGNVFSCSVMHEALH |
| | NHYTQKSLSLSLGK (SEQ ID No. 64) |
| Human IgG2 heavy | ASTKGPSVFPLAPCSRSTSESTAALGCLVKDYFPEPVTVSWNSGA |
| chain P01859 | LTSGVHTFPAVLQSSGLYSLSSWTVPSSNFGTQTYTCNVDHKPS |
| | NTKVDKTVERKCCVECPPCPAPPVAGPSVFLFPPKPKDTLMISRT |
| | PEVTCVWDVSHEDPEVQFNWYVDGVEVHNAKTKPREEQFNSTF |
| | RWSVLTWHQDWLNGKEYKCKVSNKGLPAPIEKTISKTKGQPRE |
| | PQVYTLPPSREEMTKNQVSLTCLVKGFYPSDISVEWESNGQPENN |
| | YKTTPPMLDSDGSFFLYSKLTVDKSRWQQGNVFSCSVMHEALHN |
| | HYTQKSLSLSPGK (SEQ ID No. 65) |
| Human light chain, | GQPKANPTVTLFPPSSEELQANKATLVCLISDFYPGAVTVAWKAD |
| lambda 1 | GSPVKAGVETTKPSKQSNNKYAASSYLSLTPEQWKSHRSYSCQV |
| P0CG04 | THEGSTVEKTVAPTECS (SEQ ID No. 66) |
| Human light chain, | GQPKAAPSVTLFPPSSEELQANKATLVCLISDFYPGAVTVAWKAD |
| lambda 2 | SSPVKAGVETTTPSKQSNNKYAASSYLSLTPEQWKSHRSYSCQV |
| P0DOY2 | THEGSTVEKTVAPTECS (SEQ ID No. 67) |
| Human light chain, | RTVAAPSVFIFPPSDEQLKSGTASWCLLNNFYPREAKVQWKVDN |
| kappa | ALQSGNSQESVTEQDSKDSTYSLSSTLTLSKADYEKHKVYACEVT |
| P01834 | HQGLSSPVTKSFNRGEC (SEQ ID No. 68) |

### Example 2

Anti-IGFBP3 mAbs Produced by Hybridoma Inhibit IGFBP3-TMEM219 Binding

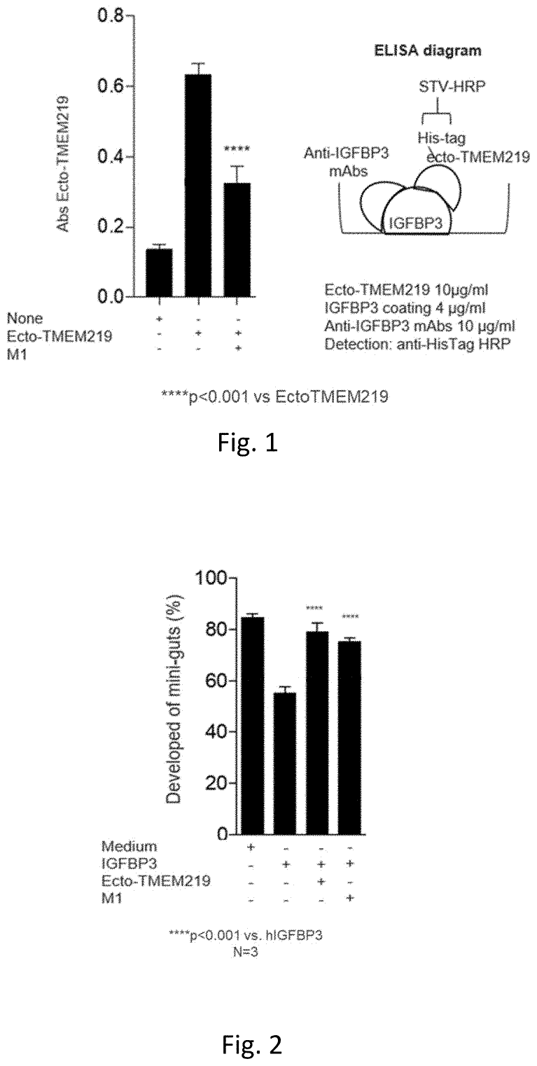

The novel anti-IGFBP3 monoclonal antibody generated using hybridoma was screened for its ability to compete with ecto-TMEM219 for the interaction with IGFBP3 using a competitive ELISA binding assay. IGFBP3, ecto-TMEM219 and the available antibodies were all used in a 1:1 ratio. The anti-IGFBP3 mAb was able to inhibit the IGFBP3-Ecto-TMEM219 (FIG. 1). This demonstrates that the anti-IGFBP3 mAb of the invention may inhibit the binding of IGFBP3 to the native TMEM219 receptor and may mimic the neutralizing activities of the ecto-TMEM219 protein.

Newly Generated Monoclonal Anti-IGFBP3 Antibodies Rescue IGFBP3-Damage in the Mini-Guts Assay in Humans.

The newly generated monoclonal Antibody was also tested in the mini-gut assay. Briefly, mini-guts were generated from crypts obtained from healthy controls (n=3) and cultured for 8 days in presence of IGFBP3 and treated with either ecto-TMEM219 or newly generated anti-IGFBP3 mAb at a ratio of 1:1 (mAbs/ecto-TMEM219: IGFBP3).

We observed that Anti-IGFBP3 mAb was comparable to ecto-TMEM219 in rescuing the negative effects of IGFBP3 on self-renewal ability (% development) and morphology (absence of crypts domain, generation of small spheroids) of large crypt organoids. (FIG. 2). This demonstrates that the anti-IGFBP3 mAb of the invention mimic the ability of the ecto-TMEM219 to rescue mini-gut growth in intestinal stem cell (ISC) injury disease conditions though preventing the binding of IGFBP3 to TMEM219.

Newly Generated Monoclonal Anti-IGFBP3 Antibodies Rescue IGFBP3-Damage on ISCs Markers

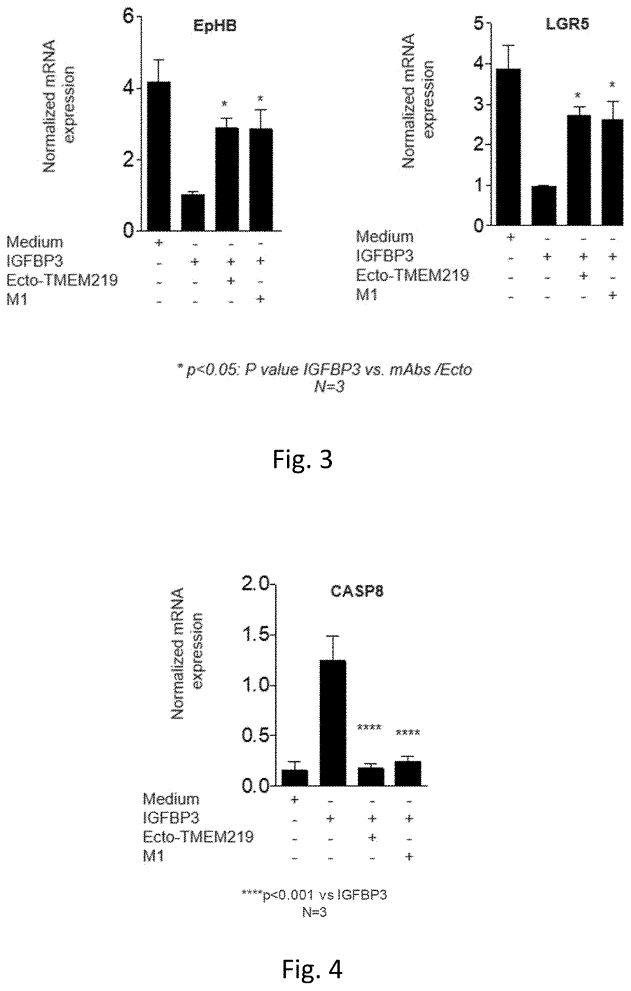

Newly generated anti-IGFBP3 mAbs, which were shown to be effective in promoting mini-guts development upon IGFBP3 exposure were also able to restore the expression of ISCs markers EphB2 and LGR5 (FIG. 3). IGFBP3-detrimental effects on ISCs are Caspase-8 mediated. The anti-IGFBP3 mAb was able to inhibit the caspase-8 upregulation induced by IGFBP3 treatment, further suggesting that it exerts a protective effect on ISCs pool by blocking the IGFBP3/TMEM219 Caspase-8-mediated apoptotic injury (FIG. 4).

The Newly Discovered Anti-IGFBP3 Antibody Rescue Mini-Guts Growth in Disease Models.

In order to confirm that the newly discovered monoclonal anti-IGFBP3 antibody prevent the detrimental effects of IGFBP3 on TMEM219-expressing intestinal stem cells, the inventors further tested them in vitro in the mini-gut obtained from IBD patients.

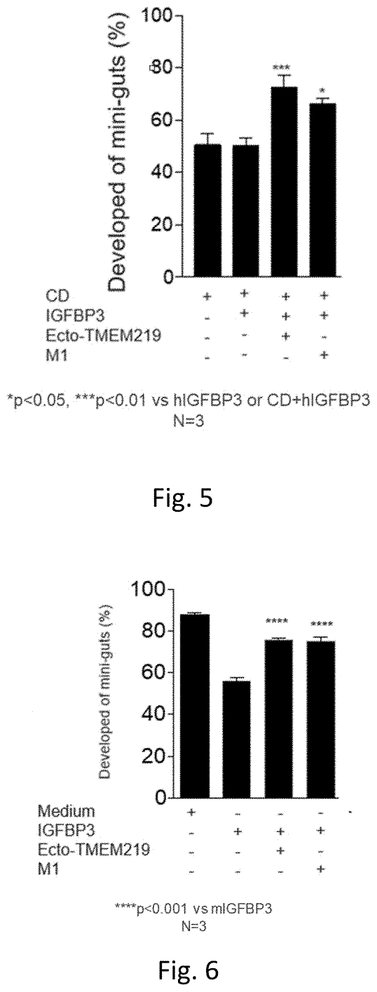

The novel anti-IGFBP3 mAb significantly improved the development of mini-guts from IBD patients of at least 20%, similarly to Ecto-TMEM219 treatment (FIG. 5).

This highlights that anti-IGFBP3 mAbs of the invention, selected for their ability to competitively inhibit ecto-TMEM binding to IGFBP3 are capable of rescuing ISCs function and preserve ISCs pool from IGFBP3-detrimental effects.

Newly Generated Monoclonal Anti-IGFBP3 Antibodies Rescue IGFBP3-Damage in Murine Mini-Guts

Crypts Isolation and Murine Mini-Guts Development

In order to confirm that the present invention antibodies have a similar tissue cross-reactivity profile in murine tissue in respect to the human tissues, the inventors further tested the monoclonal anti-IGFBP3 antibodies of the invention in the in vitro mini-gut assay in murine crypts. Crypts were obtained from control mice (n=3) (632C57BL/6J Charles River Laboratories, Lyon, France).

Isolated crypts were cultured to generate large crypts organoids namely mini-guts for 8 days in the presence of IGFBP3 with/without ecto-TMEM219. Newly generated anti-IGFBP3 mAbs were added at day 0 at a ratio of 1:1 (mAbs/ecto-TMEM219: IGFBP3).

Mini-guts development was calculated as a percentage of organoids growth after 8 days as compared to the plated isolated crypts (D′Addio F et al. Cell Stem Cell 2015 Oct. 1; 17(4): 486-498).

As shown in FIG. 6, the anti-IGFBP3 mAb rescue the negative effects of IGFBP3 on murine mini-gut self-renewal ability (% development) and morphology (absence of crypts domain, generation of small spheroids) of large crypt organoids, similarly to what is observed for ecto-TMEM219.

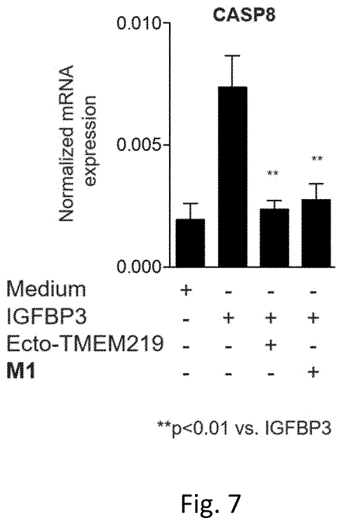

Newly generated monoclonal anti-IGFBP3 antibodies inhibit IGFBP3-mediated overexpression of caspase 8 in human beta cells IGFBP3-detrimental effects on human beta cells are Caspase-8 mediated. Interestingly, newly discovered anti-IGFBP3 mAbs were able to inhibit the caspase-8 upregulation induced by IGFBP3 treatment by at least 50% when compared to samples treated only with IGFBP3 (FIG. 7).

These results suggest that the discovered anti-IGFBP3 mAbs exert a protective effect on human beta-cells by blocking the IGFBP3/TMEM219 Caspase-8-mediated apoptotic injury.

### Example 3

Newly Generated Monoclonal Anti-IGFBP3 Antibodies Rescue IGFBP3-Damage in the Mini-Guts Assay in Humans.

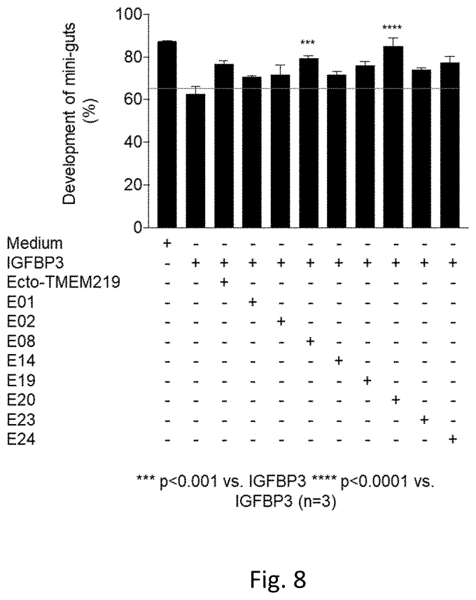

Anti-IGFBP3 monoclonal antibodies were tested in the mini-gut assay. Briefly, mini-guts were generated from crypts obtained from healthy controls (n=3) and cultured for 8 days upon IGFBP3 exposure and treated with anti-IGFBP3 mAbs at a ratio of 1:1 (mAbs: IGFBP3). Inventors observed that among 8 mAbs, E08 and E20 were comparable to ecto-TMEM219 in rescuing the self-renewal ability of large crypt organoids in the presence of IGFBP3, thus supporting a relevant effect in preventing IGFBP3-mediated damage on local stem cells (FIG. 8).

Newly Generated Monoclonal Anti-IGFBP3 Antibodies Rescue IGFBP3-Damage on ISCs Markers

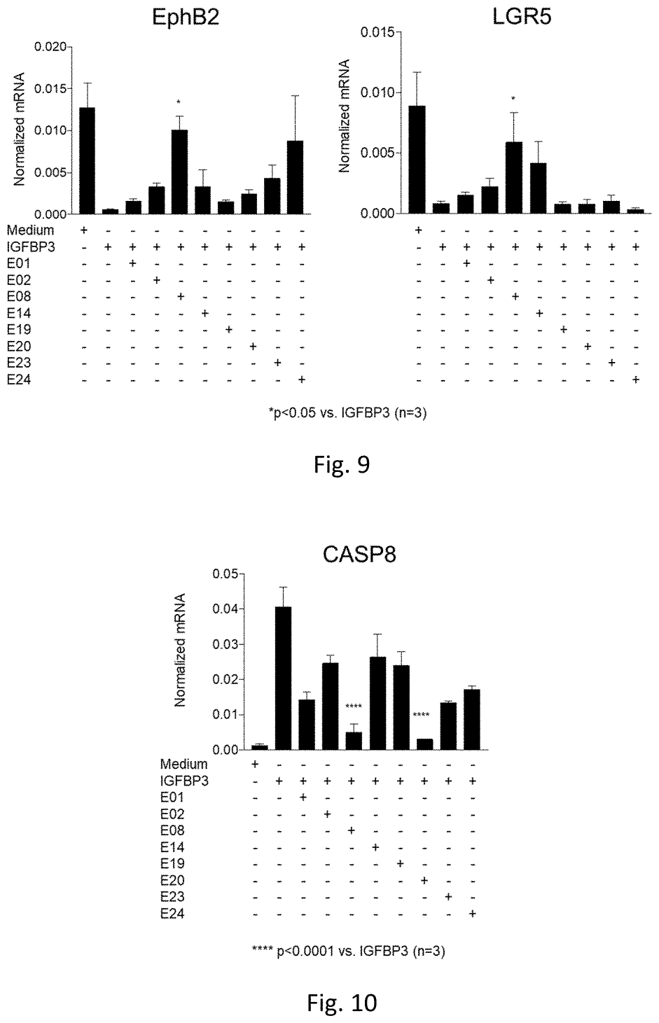

Anti-IGFBP3 mAbs, which showed to be effective in promoting mini-guts development, are also able to restore the expression of ISCs markers EphB2 and LGR5 (FIG. 9). This effect was Caspase 8-mediated as Caspase 8 expression was downregulated upon exposure to E08 and E20, further supporting that these anti-IGFBP3 mAbs exert a protective effect on the ISCs pool by blocking the IGFBP3/TMEM219 Caspase-8-mediated apoptotic injury (FIG. 10).

Newly Generated Monoclonal Anti-IGFBP3 Antibodies Rescue IGFBP3-Damage in Murine Mini-Guts

Crypts Isolation and Murine Mini-Guts Development

In order to confirm that the present invention antibodies have a similar tissue cross-reactivity profile in murine tissue in respect to the human tissues, the inventors further tested the monoclonal anti-IGFBP3 antibodies of the invention in the in vitro mini-gut assay in murine crypts. Crypts were obtained from control mice (n=3) (632C57BL/6J Charles River Laboratories, Lyon, France).

Isolated crypts were cultured to generate large crypts organoids namely mini-guts for 8 days in the presence of IGFBP3 with/without ecto-TMEM219. Newly generated anti-IGFBP3 mAbs were added at day 0 at a ratio of 1:1 (mAbs/ecto-TMEM219: IGFBP3). Mini-guts development was calculated as a percentage of organoids growth after 8 days as compared to the plated isolated crypts (D′Addio F et al. Cell Stem Cell 2015 Oct. 1; 17(4): 486-498).

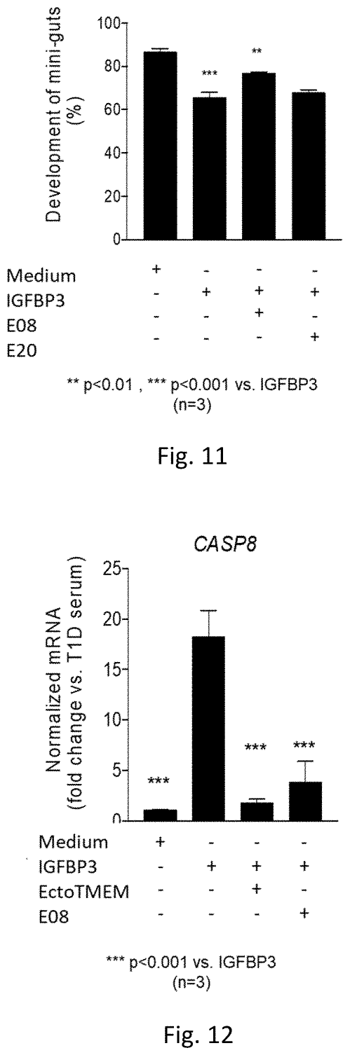

As shown in FIG. 11, antibody E08 rescued mini-guts growth in the presence of IGFBP3 and is a relevant candidate for further testing.

Anti-IGFBP3 mAbs Protect a Beta Cell Line from Apoptosis In Vitro

In order to confirm that the anti-IGFBP3 mAbs prevent the pro-apoptotic effects of IGFBP3 on TMEM219-expressing cells within the pancreas, inventors further tested them in vitro in a human beta cell line, Betalox-5. Exposure of beta cells to pooled T1 D serum increased CASP8 expression and anti-IGFBP3 mAb E08 was able to counterbalance this effect, thus supporting the beneficial effects of the newly generated monoclonal anti-TMEM219 antibodies in preventing pancreatic beta cells apoptosis (FIG. 12).

### Example 3: T1D Mouse Model

As shown in FIG. 14, the inventors assessed whether a 10 day-administration of newly generated anti-IGFBP3 mAb may prevent clinical diabetes onset in NOD mice, a mouse model selective to study autoimmune type 1 diabetes (T1 D). Anti-IGFBP3 mAbs administered intraperitoneally maintained blood glucose level under control over time and delayed onset of diabetes in T1 D NOD mouse model, with 80% of treated mice being free from diabetes at week 24 as compared to 50% of untreated controls.

Next, pancreatic tissue sections of NOD mice from untreated mice, M1S and Ecto-TMEM treated groups were analyzed at 24 weeks of age and demonstrated a reduction in islet infiltrate, with a slight increased detection of insulin positive cells as compared to untreated controls (FIG. 15).

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

### REFERENCES

1. Baxter R C. J Cell Commun Signal. 2013; 7(3):179-89.
2. Oh Y, et al., Prog Growth Factor Res. 1995; 6(2-4):503-12.
3. Ingermann A R, et al. JBC. 2010; 285(39):30233-46.
4. D′Addio F, et al. Cell stem cell. 2015; 17(4):486-98.
5. Brennand K, and Melton D. J. of cellular and molecular med. 2009; 13(3):472-87.
6. Yi P, Park J S, and Melton D A. Cell. 2014; 159(3):467-8.
7. Ben Nasr M, et al., Pharmacological res.: the official journal of the Italian Pharmacological Society. 2015; 98:31-8.
8. Keenan H A, et al. Diabetes. 2010; 59(11):2846-53.
9. Meier J J, et al., Diabetologia. 2005; 48(11):2221-8.
10. Atkinson M A, et al., Diabetes care. 2015; 38(6):979-88.
11. Nguyen K H, et al., Endocrinology. 2011; 152(6):2184-96.
12. Yakar S, et al., FASEB J. 2009; 23(3):709-19.
13. Drogan D, et al., Am J Epidemiol. 2016; 183(6):553-60.
14. Peet A, et al., Eur J Endocrinol. 2015; 173(2):129-37.
15. Kaplan G G. Nat Rev Gastroenterol Hepatol. 2015; 12(12):720-7.
16. Cui S, and Chang P Y. World J Gastroenterol. 2016; 22(31):7099-110.
17. Yancu D, et al., J Gastroenterol Hepatol. 2017; 32(1):146-53.
18. Jung P, et al., Nature Medecine 2011; 17:1225-1227.
20. George M J, et al., Curr Diab Rep 2013; 13(1):72-80.
21. Marsha J D. Am Health Drug Benefits 2011; 4(5): 312-322
22. Dhingra A K et al., Antiinflamm Antiallergy Agents Med Chem. 2015; 14(2):81-97.
23. Pithadia A B, SunitaJ. Pharmacoligal rep 2011; 63: 629-642
24. Zhe Wang, et al., Expert Opin Drug Deliv. 2010 February; 7(2):159-71
25. Sumit G, Wei W, Tsutomu and Satoshi O, Antibodies 2013; 2: 452-500;
26. Beck A, et al., Nat Rev Imm 2010; 10: 345-352
27. Huch M, et al., Nature 2013; 494 (7436): 247-250
28. Mahé M M, et al., Curr Protoc Mouse Biol 2013; 3: 217-240
29. Shimkets R A, Gene Expression Profiling. Meth. in Mol. Biology; 258. Humana Press
30. Raghavachari N, et al., Gene Expression Analaysis, Meth. in Mol. Biol; 1783. Humana Press
31. Kurien B T, et al., Western Blotting, Methods in Molecullar Biology; 1312. Humana Press
32. Hnasko R, ELISA, Methods in Molecular Blology, 1318. Humana Press
33. Lipton M S, et al., Mass Spec. of Proteins and Peptides, Meth. in Mol. Biol., 492. Humana Press
34. Brady H, Apoptosis Meth. and Protocols, Meth. in Mol. Biol., 282. Humana Press
35. Cheryl L et al., Protein-Protein interactions, Meth. in Mol. Biol., 1278. Humana Press

1. An isolated antibody or antigen binding fragment thereof that binds to human IGFBP3 with an affinity constant lower than or equal to 1.1×10⁻⁹ M and which inhibits or reduces the binding of IGFBP3 to TMEM219.

2. The isolated antibody or antigen binding fragment thereof according to claim 1 that inhibits, reduces, or neutralizes the activation of the TMEM219 receptor induced by IGFBP3.

3. The isolated antibody or antigen binding fragment thereof according to claim 1 that is effective in controlling blood glucose levels in an in vivo model.

4. The isolated antibody or antigen binding fragment thereof according to claim 1 that has at least one activity selected from:
(a) increase in IGFBP3 treated healthy subject mini-gut growth;
(b) increase in IBD-patient mini-gut growth;
(c) increase in diabetic enteropathy serum treated healthy subject mini-gut growth;
(d) increase in expression of EphB2 and/or LGR5 in IGFBP3 treated healthy subject mini gut;
(e) decrease in caspase 8 expression in IGFBP3 treated healthy subject mini-gut;
(f) decrease in β-cell loss in IGFBP3 treated β-cell;
(g) increase in expression of insulin in IGFBP3 treated β-cell;
(h) decrease in apoptosis of β-cell in IGFBP3 treated β-cell;
(i) decrease in caspase 8 expression in IGFBP3 treated β-cell;
(j) decrease in insulitis score in an animal model of diabetes; and
(k) decrease in diabetes onset in an animal model of diabetes.

5. The isolated antibody or antigen binding fragment thereof according to claim 4 wherein the increase in (a), (b), and (c) and is by at least 20%; the increase in (d) and (e) is by at least 50%; the decrease in (f) and the increase in (g) is by at least 10%.

6. The isolated antibody or antigen binding fragment thereof according to claim 1 comprising:
(a) a heavy chain variable domain (VH) comprising:
(i) a CDR1 sequence of the amino acid sequence selected from the group consisting of: SEQ ID NO: 1, 4, 7 or 9;
(ii) a CDR2 sequence of the amino acid sequence selected from the group consisting of: SEQ ID NO: 2, 5, 8 or 10; and
(iii) a CDR3 sequence of the amino acid sequence selected from the group consisting of: SEQ ID NO: 3, 6 or 11; and/or
(b) a light chain variable domain (VL) comprising:
(i) a CDR1 sequence of the amino acid sequence selected from the group consisting of: SEQ ID NO: 12, 15, 17, 20, 23, 25 or 27;
(ii) a CDR2 sequence of the amino acid sequence selected from the group consisting of: SEQ ID NO: 13, 18 or 21; and
(iii) a CDR3 sequence of the amino acid sequence selected from the group consisting of: SEQ ID NO: 14, 16, 19, 22, 24 or 26.

7. The isolated antibody or antigen binding fragment thereof according to claim 6 comprising the CDRs as indicated in Table 2 and/or in Table 3, including Table 3.1.

8. The isolated antibody or antigen binding fragment thereof according to claim 1 comprising:
(a) SEQ ID NO: 9 and SEQ ID NO: 10 and SEQ ID NO: 11 and SEQ ID NO: 27 and SEQ ID NO: 18 and SEQ ID NO: 26 or Kabat, IMGT, Chothia, AbM, or Contact CDRs of M1; or
(b) SEQ ID NO: 4 and SEQ ID NO: 5 and SEQ ID NO: 6 and SEQ ID NO: 12 and SEQ ID NO: 13 and SEQ ID NO: 14 or Kabat, IMGT, Chothia, AbM, or Contact CDRs of E08; or
(c) SEQ ID NO: 4 and SEQ ID NO: 5 and SEQ ID NO: 6 and SEQ ID NO: 23 and SEQ ID NO: 18 and SEQ ID NO: 24 or Kabat, IMGT, Chothia, AbM, or Contact CDRs of E20.

9. The isolated antibody or antigen binding fragment thereof according to claim 1 comprising:
(a) a heavy chain variable domain (VH) comprising:
(i) a CDR1 sequence of the amino acid sequence selected from the group consisting of a sequence as defined using abysis tool analysis (abysis.org);
(ii) a CDR2 sequence of the amino acid sequence selected from the group consisting of a sequence as defined using abysis tool analysis (abysis.org); and
(iii) a CDR3 sequence of as defined using abysis tool analysis (abysis.org); and/or
(b) a light chain variable domain (VL) comprising:
(ii) CDR1 sequence of the amino acid sequence selected from the group consisting of a sequence as defined using abysis tool analysis (abysis.org);
(ii) a CDR2 sequence of the amino acid sequence selected from the group consisting of a sequence as defined using abysis tool analysis (abysis.org); and
(iii) a CDR3 sequence of the amino acid sequence selected from the group consisting of a sequence as defined using abysis tool analysis (abysis.org).

10. The isolated antibody or antigen binding fragment thereof according to claim 1 comprising:
(a) a heavy chain variable domain sequence of the amino acid sequence selected from the group consisting of: SEQ ID NO:28 to SEQ ID NO:36; or
(b) a light chain variable domain sequence of the amino acid sequence selected from the group consisting of: SEQ ID NO: 37 to SEQ ID NO:45; or
(c) the light chain variable domain of (a) and the heavy chain variable domain of (b).

11. The isolated antibody or antigen binding fragment thereof according to claim 1, wherein the antibody or antigen binding fragment is selected from the group consisting of antibody E01, E02, E08, E14, E19, E20, E23, E24 M1, or an antigen binding fragment thereof.

12. An isolated antibody or antigen binding fragment thereof that:
(a) binds specifically to an epitope on IGFBP3, that is the same or similar epitope as the epitope recognized by the monoclonal antibody E01, E02, E08, E14, E19, E20, E23, E24, or M1 comprising the sequences as defined in Tables 2-7; or
(b) cross-competes for binding with the monoclonal antibody E01, E02, E08, E14, E19, E20, E23, E24, or M1 comprising the sequences as defined in Tables 2-7; or
(c) shows the same or similar binding affinity or specificity, or both, as any of antibody E01, E02, E08, E14, E19, E20, E23, E24, or M1 comprising the sequences as defined in Tables 2-7; or
(d) has one or more biological properties of an antibody chosen from any of E01, E02, E08, E14, E19, E20, E23, E24, or M1 comprising the sequences as defined in Tables 2-7; or
(e) has one or more pharmacokinetic properties of an antibody molecule described herein, wherein the antibody is any of E01, E02, E08, E14, E19, E20, E23, E24 or M1 comprising the sequences as defined in Tables 2-7.

13. The isolated antibody or antigen binding fragment thereof according to claim 1, wherein the antibody or antigen binding fragment thereof is a human or a humanized antibody.

14. The isolated antibody or antigen binding fragment thereof according to claim 1, wherein the antibody or antigen binding fragment thereof is an IgG2 or IgG4 antibody.

15. An isolated polynucleotide comprising at least one sequence that encodes the antibody or antigen binding fragment thereof according to claim 1.

16. A vector comprising the polynucleotide according to claim 15, optionally wherein the vector is selected from the group consisting of a plasmid, a viral vector, a non-episomal mammalian vector, an expression vector, and a recombinant expression vector.

17. An isolated cell comprising the polynucleotide according to claim 15, optionally wherein the isolated cell is a hybridoma or a Chinese Hamster Ovary (CHO) cell or a Human Embryonic Kidney (HEK293) cell.

18. A method of treating a disorder, comprising:
administering a therapeutically effective amount of the antibody or antigen binding fragment thereof according to claim 1 optionally wherein the disorder is selected from: diabetes, intestinal and/or bowel disorder, malabsorption syndrome, cachexia or diabetic enteropathy.

19. A pharmaceutical composition comprising the isolated antibody or antigen binding fragment thereof according to claim 1, and a pharmaceutically acceptable carrier, optionally wherein for use in the treatment of: diabetes, intestinal and/or bowel disorder, malabsorption syndrome, cachexia or diabetic enteropathy, optionally wherein the intestinal and/or bowel disorder is inflammatory bowel disease, celiac disease, ulcerative colitis, Crohn's disease or intestinal obstruction.

20. A method of inhibiting the binding of IGFBP3 to TMEM219 receptor, comprising contacting IGFBP3 with the antibody or the composition according to claim 1.

21. The isolated antibody or antigen binding fragment thereof according to claim 14, wherein the antibody or antigen binding fragment thereof is a human IgG2 or human IgG4.

22. The isolated polynucleotide according to claim 15, wherein said polynucleotide is a cDNA.

23. An isolated cell comprising the vector according to claim 16, wherein the isolated cell is a hybridoma or a Chinese Hamster Ovary (CHO) cell or a Human Embryonic Kidney cells (HEK293).

24. The isolated antibody or antigen binding fragment thereof according to claim 14, wherein the antibody or antigen binding fragment thereof is selected from the group consisting of an IgG2 kappa antibody, an IgG2 lambda antibody, an IgG4 kappa antibody and an IgG4 lambda antibody.

25. The vector according to claim 16, wherein the vector is a plasmid.

26. The vector according to claim 16, wherein the vector is a viral vector.

27. The vector according to claim 16, wherein the vector is a non-episomal mammalian vector.

28. The vector according to claim 16, wherein the vector is an expression vector.

29. The vector according to claim 16, wherein the vector is a recombinant expression vector.

30. The isolated cell according to claim 17, wherein the isolated cell is hybridoma.

31. The isolated cell according to claim 17, wherein the isolated cell is Chinese Hamster Ovary (CHO) cell.

32. The isolated cell according to claim 17, wherein the isolated cell is Human Embryonic Kidney (HEK293) cell.

33. A method of treating a disorder, comprising administering a therapeutically effective amount of a pharmaceutical composition comprising the isolated antibody or antigen binding fragment thereof according to claim 1, and a pharmaceutically acceptable carrier, optionally wherein for the use in the treatment of: diabetes, intestinal and/or bowel disorder, malabsorption syndrome, cachexia or diabetic enteropathy, optionally wherein the intestinal and/or bowel disorder is inflammatory bowel disease, celiac disease, ulcerative colitis, Crohn's disease or intestinal obstruction.

34. The method of treating a disorder of claim 18, the diabetes disorder, wherein it is Type I or Type II.

35. The method of treating a disorder of claim 18, the intestinal and/or bowel disorder, wherein it is inflammatory bowel disease, celiac disease, ulcerative colitis, Crohn's disease or intestinal obstruction.

36. The isolated antibody or antigen binding fragment thereof according to claim 1 wherein the heavy chain constant region is a human IgG4 including a Ser to Pro substitution at position 228 and a Leu to Glu substitution at position 235.
